(19) United States
(12) Patent Application Publication
(10) Pub. No.: US 2026/0315714 A1
(43) Pub. Date: Oct. 15, 2026

(54) PRODUCTS OF MANUFACTURE AND METHODS FOR TREATING, AMELIORATING OR PREVENTING MICROBIAL INFECTIONS

(71) Applicant: Topelia Aust Limited, Five Dock (AU)

(72) Inventor: Thomas Julius Borody, Five Dock (AU)

(21) Appl. No.: 19/462,448

(22) Filed: Jan. 28, 2026

Related U.S. Application Data
(63) Continuation of application No. 17/921,097, filed on Apr. 23, 2021, now abandoned.
(60) Provisional application No. 63/015,294, filed on Apr. 24, 2020.
(60) Provisional application No. 63/019,813, filed on May 4, 2020.
(60) Provisional application No. 63/060,508, filed on Aug. 3, 2020.
(60) Provisional application No. 63/109,242, filed on Nov. 3, 2020.
(60) Provisional application No. 63/123,416, filed on Dec. 9, 2020.

(51) Int. Cl. A61M 15/08; A61K 45/06; A61M 15/00; A61M 15/02

(52) U.S. Cl. CPC A61M 15/08; A61K 45/06; A61M 15/0003; A61M 15/02

(57) ABSTRACT

Provided are products of manufacture fabricated or manufactured as medical devices such as an inhaler, an ionizer, an asthma “puffer-like” device, a nebulizer or a nasal spray device, a respirator or a ventilator, a warm or a hot air delivery device, and/or a CPAP (continuous positive air pressure) device (for example, for managing or treating sleep apnea) or equivalent, for delivering one or a combination of medications or drugs, and optionally also warm or hot air, and optionally also ionized air. Products of manufacture are used for treating, preventing, ameliorating, slowing the progress of, decreasing the severity of or preventing a microbial infection, such as bacterial and viral infections, including the common respiratory viruses such as influenza virus, respiratory syncytial virus, picornavirus, parainfluenza virus, adenovirus, rhinovirus, human metapneumovirus, hantaviruses, enterovirus, a coronavirus infection, or a COVID-19 or a 2019-nCoV (or so-called Wuhan coronavirus) infection.

### RELATED APPLICATIONS

This new United States patent application is a continuation of U.S. patent application Ser. No. 17/921,097, filed Oct. 24, 2022, now pending, which is a national phase filing under 35 U.S.C. § 371 of International (PCT) Patent Application No. PCT/AU2021/050369, filed on Apr. 23, 2021, which claims the benefit of and priority to U.S. provisional patent application Ser. No. 63/015,294, filed Apr. 24, 2020, and U.S. provisional patent application Ser. No. 63/019,813 filed May 4, 2020, and U.S. provisional patent application Ser. No. 63/060,508 filed Aug. 3, 2020, and U.S. provisional patent application Ser. No. 63/109,242 filed Nov. 3, 2020, and U.S. provisional patent application Ser. No. 63/123,416 filed Dec. 9, 2020. The aforementioned applications are expressly incorporated herein by reference in their entirety and for all purposes.

### FIELD

This invention generally relates to infectious respiratory diseases and medical devices. In alternative embodiments, provided are products of manufacture fabricated or manufactured as medical devices such as an inhaler, asthma “puffer-like” device, ionizer, nebulizer or nasal spray device, a respirator or ventilator, a warm or hot air delivery device, and/or a CPAP (continuous positive air pressure) device (for example, for managing or treating sleep apnea) or equivalent, for delivering one or a combination of medications or drugs, and optionally also warm or hot air, and/or ionized air. Products of manufacture are used for treating, preventing, ameliorating, slowing the progress of, decreasing the severity of or preventing a microbial infection, such as bacterial and viral infections, including the common respiratory viruses such as influenza virus, respiratory syncytial virus, parainfluenza virus, adenovirus, rhinovirus, human metapneumovirus, hantaviruses, enterovirus, a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, products of manufacture as provided herein administer combinations, or cocktails, of a drug or drugs by inhalation, for example, by use of aerosol, mist, liquid or powder formulations for inhalation.

### BACKGROUND

Coronavirus infections have previously caused SARS (Severe Acute Respiratory Syndrome) and MERS (Middle East Respiratory Syndrome) and are particularly difficult to treat with anti-viral agents, and single drug regimens have not been found to be effective against the current coronavirus infection (2019-nCoV). The coronavirus infection (2019-nCoV), which started in China in December 2019, has spread rapidly throughout the world and has claimed hundreds of lives in China, the United States, Italy, Spain, England and numerous other nations. The known coronavirus anti-infective agents used alone are unable to cure the infection.

### SUMMARY OF INVENTION

In alternative embodiments, provided are products of manufacture fabricated or manufactured as medical devices such as an inhaler, an asthma “puffer-like” device, an ionizer, a nebulizer, a nasal spray device, a respirator or a ventilator, a warm or hot air delivery device, and/or a CPAP (continuous positive air pressure) device (for example, for managing or treating sleep apnea) or equivalent, for delivering one or a combination of medications or drugs.

In alternative embodiments, the inhaler, asthma “puffer-like” device, nebulizer or nasal spray device or equivalent is a hand-held or otherwise portable device.

In alternative embodiments, the device is a metered (for drug or medication administration) or has a dose-counting inhaled or aerosol mechanism for drugs or medications.

In alternative embodiments, the medical device further comprises a cassette, packette, interchangeable disk (for example, for holding a powder) or reservoir (optionally a refillable reservoir) in or on the product of manufacture, or a removable cassette or packette, interchangeable disk (for example, for holding a powder or powders) that can be inserted into a slot or port on the product of manufacture, or a separate reservoir or container operatively linked or joined to the product of manufacture, that comprises the drug or the medication.

In alternative embodiments, a product of manufacture, for example, medical device, as provided herein, for example, respirator or ventilator, CPAP, an inhaler, asthma “puffer-like” device, nebulizer or nasal spray device or equivalent:
is designed, manufactured or configured to deliver at least one powder (for example, a freeze-dried powder) or a liquid aerosol or mist,
   and optionally the cassette or packette, interchangeable disk (for example, for holding a powder) comprises an adjustable value that modulates or regulates the amount of the medication or drug delivered to a user of the medical device, or
   the product of manufacture, for example, medical device, comprises at least one air filter, optionally a high-efficiency particulate air (HEPA) filter or a high-efficiency particulate absorbing and high-efficiency particulate arrestance air filter, or an air filter capable of filtering out airborne viruses, or
   the product of manufacture, for example, medical device, further comprises an air ionizer (for example, a technical ionizers or a bioionizer), or a device capable of making or generating electrons and/or negatively-charged oxygen ions and/or positively-charged ions, and this air ionizer or device capable of generating electrons and/or ions is operatively connected to the product of manufacture (for example, medical device as provided herein, for example, respirator or ventilator, CPAP, an inhaler, asthma “puffer-like” device, nebulizer or nasal spray device or equivalent) such that the ionized air, or air comprising the electrons and/or negatively-charged oxygen ions and/or positively-charged ions, is delivered to a patient, optionally as a forced air or positive air pressure flow of air, to or into the nose and or mouth, and optionally to the trachea, bronchi or lungs, of a user, and optionally the air ionizer, or a device capable of making or generating electrons and/or negatively-charged oxygen ions and/or positively-charged ions, is connected to the inhalation port of a ventilator as provided herein.

In alternative embodiments, provided are products of manufacture fabricated or manufactured as medical device capable of delivering to a patient hot or warm air, optionally as a forced air or positive air pressure flow of air, to or into the nose and or mouth, and optionally to the trachea, bronchi or lungs, of a user,
wherein optionally the medical device is fabricated or manufactured as a hand-held device, and optionally the medical device is fabricated or manufactured as a respirator or ventilator, a CPAP (continuous positive air pressure) device (optionally for managing or treating sleep apnea), an inhaler, an asthma puffer or a nebulizer,
and optionally the product of manufacture delivers the forced air or positive air pressure flow of air, or warm or hot air, or vapor or steam, or room temperature air, directly into the user's mouth and/or nose, and optionally the forced air or positive air pressure flow of air, and/or the room temperature, hot or warm air, is delivered or permeates through the nose into the sinuses and turbinates, down the trachea, and into the main bronchi and bronchioles,
and optionally the product of manufacture comprises controls or dials to set or adjust the warmth or heat of the air and/or to adjust the force and/or the volume of air that is delivered into the nose and/or mouth, and/or bronchi and lungs of the user,
and optionally the product of manufacture is battery powered,
and optionally the product of manufacture comprises a mouthpiece or facemask (optionally a disposable mouthpiece or facemask) to fit or place the device over the mouth and/or nose for forced air delivery, and optionally the mouthpiece or facemask can fit over both the nose and the mouth for simultaneous air delivery to or into both nose and mouth,
and optionally the product of manufacture comprises a refillable, renewable and/or disposable cassette, packette, interchangeable disk (for example, for holding a powder) or drug-comprising module disposed onto or in the medical device, or a removable cassette or packette, interchangeable disk (for example, for holding a powder) that can be inserted into a slot or port on the product of manufacture, or a separate reservoir or container operatively linked or joined to the product of manufacture, and optionally the refillable, renewable and/or disposable cassette, packette, interchangeable disk (for example, for holding a powder) or drug-comprising module comprises a drug or the medication, and optionally the drug or medication comprises a formulation or a drug combination as provided herein,
and optionally the drug or medication comprises an asthma or an anti-microbial (for example, antiviral, antibacterial or antifungal) drug, or an antibiotic agent or drug (optionally gentamycin, lincomycin, kanamycin, streptomycin, vancomycin, or others), a biofilm dissolving agent (optionally PULMOZYME™), an anticholinergic (optionally tiotropium bromide or SPIRIVA™), a bronchodilator, steroid, a corticosteroid, a antileukotriene, or an asthma drug (optionally formoterol, salbutamol, albuterol, or VENTOLIN™), an antifungal agent, itraconazole (for example, SPORANOX™, SPORAZ™, ORUNGAL™), an anti-inflammatory agent or any formulation or drug combination as provided herein,
and optionally the antibacterial antibiotic comprises an antimycobacterial drug, and optionally the antimycobacterial drug comprises clofazimine (optionally LAMPRENE™), optionally dosaged at about 100 mg per day, or between about 50 mg and 150 mg per day, and optionally also including colchicine,
and optionally clofazimine, or LAMPENE™, optionally dosaged at about 100 mg per day, or between about 50 mg and 150 mg per day, and chloroquine (or ARALEN™), chloroquine phosphate, chloroquine diphosphate and/or hydroxychloroquine (HCQ) (optionally, PLAQUENIL™), optionally also comprising zinc (optionally a zinc sulphate, acetate, gluconate or picolinate, or zinc oxide nanoparticles, optionally at a dosage of between about 1 mg to 250 mg, or about 50 mg per day), and optionally also including colchicine;
and optionally the product of manufacture comprises at least one air filter, optionally a high-efficiency particulate air (HEPA) filter or a high-efficiency particulate absorbing and high-efficiency particulate arrestance air filter, or an air filter capable of filtering out airborne viruses.

In alternative embodiments, products of manufacture as provides herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising one or any of the following in any combination:
(a) opaganib or YELIVA™; or opaganib or YELIVA™ and oral and/or inhaled or aerosol chloroquine (or ARALEN™), chloroquine phosphate, quinine, chloroquine diphosphate and/or hydroxychloroquine (for example, PLAQUENIL™); or opaganib, hydroxychloroquine and azithromycin (for example, ZITHROMAX™, or AZITHROCIN™, optionally an oral extended-release formulation of azithromycin, or ZMAX™),
wherein optionally each or both of the opaganib and the chloroquine (or ARALEN™), chloroquine phosphate, quinine, chloroquine diphosphate and/or hydroxychloroquine (for example, PLAQUENIL™) and/or azithromycin are in or formulated as a formulation for inhalation, for example, formulated as an aerosol, liquid or powder, or each or both are formulated for oral, intramuscular or intravenous administration,
wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage;
(b) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 240 mg per day,
optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent),
optionally formulated as an aerosol, spray, mist, liquid or powder, optionally formulated as an aerosol, spray, mist, liquid or powder,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is formulated with and/or administered with chloroquine (or ARALEN™), chloroquine phosphate, chloroquine diphosphate and/or hydroxychloroquine (optionally, PLAQUENIL™) with or without zinc (optionally a zinc sulphate, acetate, gluconate or picolinate), and optionally this combination is administered weekly, or every two week, or one every 5 to 28 days, as a prophylactic,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered alone for 1, 2, 3, 4, 5, 6, 7, 8, 9, 10 or up to 20 or more days, followed by administration of an antibiotic (optionally doxycycline) for a corresponding period of days, and optionally repeating the cycle of dosaging,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is formulated or administered with:
   (i) at least one antibiotic (wherein optionally the antibiotic is doxycycline (optionally, DORYX™, DOXYHEXA™, DOXYLIN™) (optionally formulated or administered at a dosage of between about 25 mg to 600 mg per dose or per day), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
   (ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
   (iii) a zinc (optionally a zinc sulphate, acetate, gluconate or picolinate) optionally formulated or administered at a dosage of between about 1 mg to 250 mg; and/or
   (iv) at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered or formulated alone or in combination with any of the above (i) to (iv) (for example, at least one antibiotic, chloroquine (or ARALEN™), chloroquine phosphate, chloroquine diphosphate and/or hydroxychloroquine (optionally, PLAQUENIL™), zinc and/or at least one vitamin are formulated (and administered) as oral formulations (for example, as tablets, capsules, gels or geltabs), injectable formulations, powders (for example, for inhalation or for addition to an ingestible liquid) or liquids (for example, for ingestion, infusion or injection);
(c) lopinavir, ritonavir (optionally NORVIR™) and oseltamivir (for example, TAMIFLU™), and/or zanamivir (RELENZA™);
(d) lopinavir combined (formulated) with ritonavir (optionally NORVIR™), or KALETRA™, ALTERA™, ALUVIA™, KALMELTREX, or LOPIMUNE™, and/or zanamivir (RELENZA™), and optionally the lopinavir, ritonavir and/or zanamivir are separately formulated;
(e) lopinavir combined (formulated) with ritonavir ((optionally NORVIR™), KALETRA™, ALTERA™, ALUVIA™, KALMEL TREX, LOPIMUNE™), or lopinavir and ritonavir, and oseltamivir (for example, TAMIFLU™), and/or zanamivir (RELENZA™), optionally also with inhaled or aerosol formulations or versions of chloroquine (or ARALEN™), chloroquine phosphate, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) simultaneously;
(f) lopinavir, ritonavir (optionally NORVIR™), chloroquine and oseltamivir (or TAMIFLU™), and/or zanamivir (RELENZA™); wherein optionally the chloroquine comprises inhaled or aerosol chloroquine (or ARALEN™), chloroquine phosphate, quinine, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) simultaneously;
(g) lopinavir and oseltamivir (for example, TAMIFLU™), and/or zanamivir (RELENZA™)
(h) ritonavir (optionally NORVIR™) and oseltamivir (for example, TAMIFLU™), and/or zanamivir (RELENZA™);
(i) remdesivir (for example, GS-5734™, Gilead Sciences) alone, or oseltamivir (for example, TAMIFLU™) and remdesivir (for example, GS-5734™, Gilead Sciences), and optionally the remdesivir and/or oseltamivir is an oral formulation and/or an inhaled or aerosol remdesivir or oseltamivir formulation;
(j) oseltamivir (for example, TAMIFLU™) and efavirenz (for example, SUSTIVA™), and/or zanamivir (RELENZA™);
(k) oseltamivir (for example, TAMIFLU™) and nevirapine (or the combination efavirenz (optionally, SUSTIVA™) with emtricitabine and tenofovir (optionally tenofovir alafenamide or tenofovir disoproxil or VIREAD™), or ATRIPLA™);
(l) oseltamivir (or TAMIFLU™) and amprenavir (for example, AGENERASE™);
(m) oseltamivir (for example, TAMIFLU™) and nelfinavir (for example, VIRACEPT™); or
(n) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (for example, nitazoxanide or tizoxanide) and oseltamivir (or TAMIFLU™);
(o) plitidepsin (also known as dehydrodidemnin B), or APLIDIN™ (PharmaMar, S.A.);
(p) an inhibitor or S-phase kinase-associated protein 2 (SKP2), or dioscin, or niclosamide, or NICLOCIDE™, FENASAL™, or PHENASAL™.
(q) ribavirin or tribavirin (or COPEGUS™, REBETOL™, or VIRAZOLE™), interferon beta 1b, or a combination of ribavirin and interferon beta, or a combination of lopinavir and ritonavir (optionally NORVIR™) and interferon-beta-1b;
(r) abacavir, acyclovir for example, (ACICLOVIR™), adefovir, amantadine, ampligen, amprenavir (for example, AGENERASE™), aprepitant, arbidol, atazanavir, tenofovir, a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (for example, COMBVIR™), cobicstat, colisitin, cocaine, darunavir, delavirdine, descovy, didanosine, docosanol, dolutegravir, ecoliever, edoxudine, efavirenz (optionally, SUSTIVA™), elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430, IMMUCILLIN-A™), remdesivir (for example, GS-5734™, Gilead Sciences), cytarabine, gemcitabine, emtricitabine, lamivudine, zalcitabine, abacavir, acyclovir, entecavir, stavudine, telbivudine, idoxuridine and/or trifluridine or any combination thereof), oseltamivir (or TAMIFLU™), peginterferon alfa-2a, penciclovir, peramivir (for example, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir (optionally SOFORAL™, SOLVALDI™), stavudine, telaprevir, tegobuv, tenofovir (optionally tenofovir alafenamide or tenofovir disoproxil, or VIREAD™), tipranavir, trifluridine, trizivir, tromantadine, truvada, valaciclovir (for example, VALTREX™), valganciclovir, valrubicin, vapreotide, vicriviroc, vidarabine, viramidine, velpatasvir, vivecon, zalcitabine, zanamivir (for example, RELENZA™), zidovudine, an immunosuppressive drug (optionally tocilizumab or atlizumab, or ACTEMRA™, or ROACTEMRA™) or any combination thereof;
(s) an mucolytic therapy or drug, optionally acetylcysteine, ambroxol, bromhexine, carbocisteine, erdosteine, mecysteine or dornase alfa, or an expectorant, optionally guaifenesin;
(t) a viral, or a coronavirus or a COVID-19, protease inhibitor, optionally ASC09 (CAS registry no. 1000287-05-7) (Janssen Research and Development, LLC), ritonavir (optionally NORVIR™) or ASC09 and ritonavir, or a JAK1/2 inhibitor (optionally baricitinib), optionally compound 11r (University of Lubeck, Germany, see for example, Zhang et al J. Med Chem 2020, Feb. 11, 2020), or darunavir, cobicistat or darunavir and cobicistat;
(u) an angiotensin-converting enzyme 2 (ACE2) inhibitor, optionally to block the site of viral spike protein interaction for anti-SARS-CoV-2 infection control;
(v) an anti-vascular endothelial growth factor (VEGF) (optionally VEGF-A) drug or antibody, optionally bevacizumab;
(w) a protease inhibitor, optionally danoprevir, optionally a serine protease inhibitor, optionally or narlaprevir (optionally ARLANSA™), camostat or camostat mesilate (or FOIPAN™);
(x) anti-PD-1 checkpoint inhibitor, optionally camrelizumab;
(y) a compound or antibody capable of binding complement factor C5 and blocking membrane attack complex formation, optionally eculizumab;
(z) a cathepsin inhibitor, optionally a cathepsin K, B or L inhibitor, optionally relacatib;
(aa) thalidomide, or thalidomide and glucocorticoid (optionally low-dose glucocorticoid), or and thalidomide and celecoxib;
(bb) an antibacterial antibiotic, a macrocyclic lactone antibiotic, or a macrolide drug,
wherein optionally the macrolide drug comprises azithromycin (for example, ZITHROMAX™, or AZITHROCIN™, optionally an oral extended-release formulation of azithromycin, or ZMAX™), clarithromycin (for example, BIAXIN™), erythromycin (for example, ERYTHROCIN™), or fidaxomicin (for example, DIFICID™ or DIFICLIR™), troleandomycin (for example, TEKMISIN™), tylosin (for example, TYLOCINE™ or TYLAN™), solithromycin (for example, SOLITHERA™), oleandomycin (or SIGMAMYCINE™), midecamycin, roxithromycin, kitasamycin or turimycin, josamycin, carbomycin or magnamycin, and/or spiramycin, and optionally the macrocyclic lactone antibiotic comprises an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally as a single or a divided dose,
and optionally the macrolide drug, optionally azithromycin, is administered at a dosage of about 100 to 200 mg a day, or at about 25 to 100 mg three times a day (tid), or at 50 mg tid,
wherein optionally the antibacterial antibiotic or the macrolide drug. optionally azithromycin, is formulated for normal release or as a delayed release formulation,
and optionally the antibacterial antibiotic comprises a tetracycline class drug a glycylcycline or a fluorocycline class drug, or an analogue thereof, and optionally the tetracycline class drug comprises one or more of: tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof;
(cc) chloroquine (or ARALEN™), chloroquine phosphate, quinine, chloroquine diphosphate and/or hydroxychloroquine (for example, PLAQUENIL™) alone or with (or formulated with) or in combination with any of (a) to (aa), or chloroquine, chloroquine phosphate, quinine, chloroquine diphosphate and/or hydroxychloroquine (for example, PLAQUENIL™) and oseltamivir (or TAMIFLU™);
(dd) chloroquine (for example, ARALEN™), chloroquine phosphate, quinine, chloroquine diphosphate and/or hydroxychloroquine (for example, PLAQUENIL™) alone or with:
   (i) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 10 mg to 80 mg dosages, or between about 6 mg to 60 mg dosages, or 12 to 60 mg dosages, and/or
   (ii) vitamin D, vitamin D2 (or ergocalciferol), vitamin D3 (or cholecalciferol) or calcifediol, optionally at a dosage of between about 3,000 to 100,000 units per day, or between about 10,000 to 50,000 units per day, or
   (iii) with (i) and (ii) and zinc (optionally a zinc sulphate, acetate, gluconate or picolinate) optionally administered at about 75 to 100 mg/day, or optionally at a dosage of between about 1 mg to 250 mg, or
   (iv) the combination of (iii) also with a tetracycline class drug, wherein optionally the tetracycline class drug comprises doxycycline, or DORYX™, DOXYHEXA™, DOXYLIN™, optionally dosages at between about 100 mg to 600 mg per day, optionally between about 200 mg to 400 mg;
(ee) colchicine, or COLCRYS™, MITIGARE™;
(ff) a corticosteroid class drug such as budesonide (optionally RHINOCORT™ or PULMICORT™), prednisolone (or ORAPRED™), methyl-prednisolone, prednisone (or DELTASONE™ or ORASONE™) or hydrocortisone (or CORTEF™),
and optionally the corticosteroid class drug (for example budesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
and optionally the corticosteroid class drug is formulated as a powder or for administration in an inhaler or by nasal spray, or for rectal administration,
and optionally the corticosteroid class drug (for example, budesonide) is administered together with or in combination with 10 mg to 80 mg, an antibiotic (optionally azithromycin or a tetracycline class drug
and optionally the corticosteroid class drug (for example budesonide) is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, an antibiotic (optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™) or a tetracycline class drug, optionally doxycycline), zinc and/or a vitamin (optionally vitamin D, D2 (or ergocalciferol), D3 (or cholecalciferol or calcifediol), C, E, B12, B6);
(gg) an anti-androgen drug, optionally bicalutamide, optionally CASODEX™, and optionally the anti-androgen, optionally bicalutamide is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
(hh) a hydrocortisone or cortisol (optionally CORTEF™, SOLUCORTEF™), optionally hydrocortisone sodium succinate or hydrocortisone acetate, or dexamethasome (optionally DEXTENZA™, OZURDEX™, NEOFORDEX™);
(ii) an alpha-ketoamide (a-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2), and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, optionally doxycycline); chloroquine (or ARALEN™), chloroquine phosphate, chloroquine (optionally, GS-5734™, Gilead Sciences); oseltamivir (or TAMIFLU™); and/or, hydrocortisone; or, any combination thereof;
(jj) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 300 mg per day,
optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated with and/or administered with chloroquine (or ARALEN™), chloroquine phosphate, chloroquine diphosphate and/or hydroxychloroquine (optionally, PLAQUENIL™) with or without zinc (optionally a zinc sulphate, acetate, gluconate or picolinate), and optionally this combination is administered weekly, or every two week, or one every 5 to 28 days, as a prophylactic,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered alone for 1, 2, 3, 4, 5, 6, 7, 8, 9 or 10 days or up to 20 more days, followed by administration of an antibiotic (optionally doxycycline) for a corresponding period of days, and optionally repeating the cycle of dosaging,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated or administered with:
   (i) at least one antibiotic (wherein optionally the antibiotic is doxycycline (optionally, DORYX™, DOXYHEXA™, DOXYLIN™) (optionally formulated or administered at a dosage of between about 25 mg to 500 mg), or azithromycin (optionally, ZITHROMAX™, or AZITHROCIN™, optionally an oral extended-release formulation of azithromycin, or ZMAX™) (optionally formulated or administered at a dosage of between an about 50 mg to 2000 mg);
   (ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
   (iii) a zinc (optionally a zinc sulphate, acetate, gluconate or picolinate), optionally administered or formulated at about 75 to 100 mg/day, or optionally formulated or administered at a dosage of between about 1 mg to 250 mg; and/or
   (iv) at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day,
   and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered or formulated alone or in combination with any of the above (i) to (iv) (for example, at least one antibiotic, chloroquine (or ARALEN™), chloroquine phosphate, chloroquine diphosphate and/or hydroxychloroquine (optionally, PLAQUENIL™), zinc (optionally a zinc sulphate, acetate, gluconate or picolinate) and/or at least one vitamin are formulated (and administered) as oral formulations (for example, as tablets, capsules, gels or geltabs), injectable formulations, powders (for example, for inhalation or for addition to an ingestible liquid) or liquids (for example, for ingestion, infusion or injection);
(kk) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™, DOXYHEXA™, DOXYLIN™;
(ll) a dendrimer, optionally astodrimer sodium (Starpharma, Melbourne, Australia);
(mm) an antihistamine class drug such as azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
(nn) a selective serotonin reuptake inhibitor (SSRI) class drug, optionally fluvoxamine, or LUVOX™, FAVERIN™, FLUVOXIN™;
(oo) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™;
(pp) any one or several or all of (a) to (oo) with (or formulated with or formulated as an) inhaled or aerosol formulation and/or formulated with or formulated as an oral, intramuscular (IM) or intravenous (IV) formulation, wherein optionally both the inhaled or aerosol and the oral, IV and/or IM formulations are administered simultaneously or sequentially,
and optionally the inhaled or aerosol formulation comprises chloroquine (or ARALEN™), chloroquine phosphate, quinine, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) administered simultaneously or overlapping; or
(qq) any combination of (a) to (pp), optionally with or also formulated with least one vitamin or mineral, wherein optionally the at least one vitamin or mineral comprises vitamin K, vitamin C (optionally administered at 500 mg bid), vitamin D, vitamin B6 (or pyridoxine) and/or vitamin B12, or zinc (optionally zinc sulfate, acetate, gluconate or picolinate, optionally administered at about 50 to 200 mg/day, or 100 mg/day), or a flavonoid, plant flavonol or quercetin optionally administered at between about 250 to 500 mg bid, or melatonin, and/or aviptadil (or INVICORP™), or
(rr) any combination of (a) to (qq),
and optionally any one of, any combination of or all of (a) to (rr) are formulated as an aerosol, a mist or a powder.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of oseltamivir, lopinavir and ritonavir. Optionally the combination of oseltamivir, lopinavir and ritonavir further comprises chloroquine or hydroxychloroquine. Optionally the combination of oseltamivir, lopinavir and ritonavir further comprises chloroquine or hydroxychloroquine, and opaganib. Optionally the combination further comprises chloroquine. Optionally the combination further comprises hydroxychloroquine. Optionally, the combination of oseltamivir, lopinavir and ritonavir further comprises zinc and bismuth.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of lopinavir, ritonavir, and chloroquine or hydroxychloroquine. Optionally the combination of lopinavir, ritonavir, and chloroquine or hydroxychloroquine further comprises remdesivir and interferon.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising:
an initial loading dose of chloroquine or hydroxychloroquine of between about 250 mg, 300 mg, 350 mg, 300 mg or 500 mg and 1.5 g, or between about 400 mg and 1 g,
optionally followed by administration of chloroquine or hydroxychloroquine every 4 to 10 days, or every 1, 2, 3, 4, 5, 6, 7, 8, 9, 10 days or up to 20 or more days, at a lower total daily dosage of between about 50 gm to 200 mg, or about 100 mg, optionally continuing for between about one week to one month,
   wherein the chloroquine or hydroxychloroquine is administered together with:
a macrolide drug, optionally azithromycin, and optionally the macrolide drug is started with a loading dose, optionally an oral, IV or IM dosage of between about 400 mg to 500 mg and 1 g, or at about 500 mg, optionally with follow up administrations every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month, and/or
opaganib, wherein optionally the opaganib is administered once a day, twice a day or three times a day at a dosage of between about 100 to 600 mg per day or per dosage, or at about 100, 200, 300, 400, 500 or 600 mg per day or per dose, and optionally the opaganib is also administered or formulated with an antibiotic, optionally azithromycin or doxycycline, ivermectin, optionally 12 mg ivermectin, optionally administered on days 1, 3, 6 and 8, hydroxychloroquine and/or zinc, optionally zinc sulfate, optionally at 50 mg daily dosage, and/or lopinavir combined with ritonavir.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of:
(1) hydroxychloroquine (optionally, PLAQUENIL™) is administered at a 400 bid (twice a day) loading dose on day one, the at 200 mg bid for the next nine or ten days;
(2) azithromycin is administered (optionally, ZITHROMAX™, or AZITHROCIN™) at a 500 mg bid loading dose on day one, then 500 mg in the morning (MANE) for days two, three and four, then azithromycin ceased and replaced by doxycycline (optionally, DORYX™, DOXYHEXA™, DOXYLIN™) 100 mg bid for the remainder of the treatment (ten or eleven days), or
   azithromycin is first administered (optionally, ZITHROMAX™, or AZITHROCIN™) at a 500 mg bid loading dose on day one, then 500 mg in the morning (MANE) for days two, three and four, then azithromycin ceased, and doxycycline 100 mg bid (or between about 25 to 500 mg bid) (optionally, DORYX™, DOXYHEXA™, DOXYLIN™) every day for the full duration of the treatment (ten or eleven days, or more); and
(3) zinc sulfate is administered at a dosage of 100 mg MANE every day of the treatment,
wherein optionally the treatment lasts between about 10 days and 3 weeks, or 11 days and 2 weeks, or for about 10, 11, 12, 13 or 14 days.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of hydroxychloroquine (optionally, PLAQUENIL™), an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; zinc (Zn); vitamin (Vit) D3; and, vitamin C, or any combination thereof, for example hydroxychloroquine, Vitamin C, Vitamin D (optionally cholecalciferol, vitamin D3 or calcifediol), and Zinc.

In alternative embodiments, products of manufacture as provides herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of hydroxychloroquine (optionally, PLAQUENIL™), azithromycin (optionally, ZITHROMAX™, or AZITHROCIN™, optionally an oral extended- or delayed-release formulation of azithromycin, or ZMAX™)), vitamin e, vitamin D (optionally cholecalciferol, vitamin D3 or calcifediol), and zinc, or any combination thereof.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising drug regimens as set forth below (Arm C and Arm D being separate exemplary treatment regimens), where the numbers are in milligrams (mgs), and each column represents a day (i.e., the first column is day 1, the last column is day 10):

| ARM C (option 1) | day 1 | day 2 | day 3 | day 4 | day 5 | day 6 | day 7 | day 8 | day 9 | day 10 |
|---|---|---|---|---|---|---|---|---|---|---|
| Hydroxycloroquine | 800 | 400 | 400 | 400 | 400 | 400 | 400 | 400 | 400 | 400 |
| Ivermectin | 12 | | | | | | 12 | | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |

or, and alternative ARM C has 12 mg ivermectin administered on days 1, 3, 6 and 8:

| ARM C (option 2) | day 1 | day 2 | day 3 | day 4 | day 5 | day 6 | day 7 | day 8 | day 9 | day 10 |
|---|---|---|---|---|---|---|---|---|---|---|
| Hydroxycloroquine | 800 | 400 | 400 | 400 | 400 | 400 | 400 | 400 | 400 | 400 |
| Ivermectin | 12 | | 12 | | | 12 | | 12 | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |

| ARMD | day 1 | day 2 | day 3 | day 4 | day 5 | day 6 | day 7 | day 8 | day 9 | day 10 |
|---|---|---|---|---|---|---|---|---|---|---|
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |
| Ivermectin | 12 | | | | | | 12 | | | |
| Bismuth | 1048 | 1048 | 1048 | 1048 | 1048 | 1048 | 1048 | 1048 | 1048 | 1048 |
| subsalicylate | | | | | | | | | | |

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising colchinine.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of colchicine and hydroxychloroquine. Optionally the combination further comprises zinc. Optionally, the combination further comprises an avermectin class antibiotic. Optionally the combination further comprises vitamin C and/or vitamin D.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of hydroxychloroquine and an antibiotic. Optionally, the combination further comprises zinc. Optionally, the combination further comprises vitamin C and/or vitamin D. Optionally the combination further comprises ivermectin. Optionally, the combination further comprises amantadine. Optionally, the antibiotic is selected from the group consisting of azithromycin, doxycycline, and clarithromycin.

In alternative embodiments, products of manufacture as provided herein, for example, medical devices, comprise and can deliver (for example, can deliver as a mist, aerosol or powder) a therapeutic combination of drugs or a drug, a pharmaceutical dosage form, a drug delivery device, or a product of manufacture, comprising a combination of cyclosporine, ivermectin, doxycycline, and zinc.

In alternative embodiments of the therapeutic combinations of the drugs or drug, pharmaceutical dosage form, drug delivery device, or product of manufacture as provided herein:
the drugs or drug, pharmaceutical dosage form, drug delivery device, or product of manufacture further comprise (for example, are formulated with, or are administered with) an (or an additional) anti-viral drug or medication, or anti-microbial drug, or palliative agent or drug,
wherein optionally the anti-viral drug or medication, or anti-microbial drug, is or comprises efavirenz (for example, SUSTIVA™), tenofovir (optionally tenofovir alafenamide or tenofovir disoproxil, or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOYALDI™, SOFORAL™); or, an adenosine analog (optionally galidesivir, optionally BCX4430, IMMUCILLIN-A™),
and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir and cobicistat (or EVOTAZ™); abacavir, lamivudine and dolutegravir (TRIUMEQ™); tenofovir (or tenofovir disoproxil or tenofovir disoproxil, or VIREAD™, or emtricitabine) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (or ATRIPLA™); lamivudine, nevirapine and stavudine (for example, TRIOMUNE™); atazanavir and cobicistat (for example, EVOTAZ™); lamivudine and raltegravir (for example, DUTREBIS™); lamivudine and dolutegravir (or DOVATO™); doravirine, lamivudine and tenofovir (for example, DELSTRIGO™); or lamivudine, zidovudine and nevirapine (for example, CUOVIR-N™), and optionally the anti-viral drug or drug combination comprises daclatasvir (optionally DAKLINZA™);
and optionally the additional anti-viral drug or medication, or anti-microbial drug, is formulated with the chloroquine (for example, ARALEN™), chloroquine phosphate, quinine, chloroquine diphosphate, hydroxychloroquine (for example, PLAQUENIL™), lopinavir, chloroquine (for example, ARALEN™), chloroquine phosphate, quinine, chloroquine diphosphate, hydroxychloroquine (for example, PLAQUENIL™), lopinavir, ritonavir and/or oseltamivir,
and optionally the anti-viral drug or medication, or anti-microbial drug, or palliative agent comprises or further comprises zinc (optionally zinc sulfate, acetate, gluconate or picolinate, optionally administered at about 50 to 200 mg/day, or 100 mg/day), and/or at least one vitamin, optionally vitamin K, vitamin D (optionally Vitamin D3, optionally administered at about 1000 to 4000 ugm/day), vitamin B6 (or pyridoxine), vitamin B12, vitamin E, and/or vitamin C (optionally administered at 500 mg bid), or a flavonoid, plant flavonol or quercetin optionally administered at between about 250 to 500 mg bid, or melatonin, which optionally is given enterally or parenterally;
   the anti-viral drug or medication is or comprises an anti-retroviral drug or drug combination are formulated or administered with an immunosuppressive drug (optionally tocilizumab or atlizumab, or ACTEMRA™, or ROACTEMRA™),
   the chloroquine (for example, ARALEN™), chloroquine phosphate, quinine, chloroquine diphosphate, hydroxychloroquine (for example, PLAQUENIL™), lopinavir, ritonavir (optionally NORVIR™) and/or oseltamivir are formulated separately or together, or the lopinavir and ritonavir are formulated together and the oseltamivir is formulated separately;
   the therapeutic combination of drugs or drug, pharmaceutical dosage form, or the chloroquine (for example, ARALEN™), chloroquine phosphate, quinine, chloroquine diphosphate, hydroxychloroquine (for example, PLAQUENIL™), lopinavir, ritonavir (optionally NORVIR™) and/or oseltamivir and/or the anti-viral drug or medication, or anti-microbial drug are formulated or contained in a liquid formulation (optionally sterile saline or water), a spray, a powder, an aerosol, or any formulation for inhalation, a pill, a capsule, a tablet, or a geltab, or equivalents;
   and optionally the therapeutic combination of drugs or drug, pharmaceutical dosage form, or the chloroquine (for example, ARALEN™), chloroquine phosphate, quinine, chloroquine diphosphate, hydroxychloroquine (for example, PLAQUENIL™), lopinavir, ritonavir (optionally NORVIR™) and/or oseltamivir and/or the anti-viral drug or medication, or anti-microbial drug, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; and/or
   the lopinavir, ritonavir (optionally NORVIR™) and oseltamivir are formulated or packaged for administration as or dosing in the ratio of 25:100:75 of lopinavir:ritonavir:oseltamivir.

In alternative embodiments, provided are drug delivery devices or packages, a kit, a blister package, a clamshell or a tray, comprising a therapeutic combination of drugs or drug, a pharmaceutical dosage form or a formulation as provided herein, wherein the drug delivery device comprises an inhalation device or inhaler or a nasal spray device, and optionally the inhaler or a nasal spray device is a hand-held inhaler or a nasal spray device, and optionally the inhaler or a nasal spray device is a metered or dose-counting inhaler or a nasal spray device, with single or numerous powders for inhalation to prevent or treat infections,
wherein the drug delivery device or package, blister pack, clamshell or tray comprises a plurality of compartments spatially arranged on the drug delivery device or package, blister pack, clamshell or tray to follow a dosage administration regimen, wherein the spatially arranged plurality of compartments are in at least two rows, each row marked for the time for which a drug of the drug combination, or the tablets, pills, capsules, geltabs or equivalents, are to be taken by a user (optionally a patient), optionally one row marked for morning, breakfast or AM administration, and one row marked for evening, dinnertime or PM administration, and optionally the row or rows marked for morning, breakfast or AM administration is or are positioned above the row or rows marked for evening, dinnertime or PM administration,
and optionally the spatially arranged plurality of compartments are in four rows, two rows marked for morning, breakfast or AM administration, and two rows marked for evening, dinnertime or PM administration,
and optionally the spatially arranged plurality of compartments are arranged to facilitate and/or direct the patient to take the drugs in that row two times a day (bid), three times a day (tid), four times a day, or up to ten times a day (wherein optionally the higher amounts are for the very sick), optionally also comprising indication of actual times for patient to ingest the drugs in each row, and if appropriate, directions to also ingest fluids or other drugs and/or food (for example, small amounts of food),
and optionally each row comprises seven compartments for one dosage administration for each day of the week, or eight compartments for one dosage administration for each day of the week and one spare, and optionally each vertically arranged set of compartments, or columns, are marked for which day of the week the dosage formulations contained therein are to be taken by the user, and optionally where the drug delivery device or package, blister pack, clamshell or tray has one row for morning, breakfast or AM administration, and one row marked for evening, dinnertime or PM administration, each column or day will have two compartments, optionally where the compartment for morning, breakfast or AM administration is above the compartment for evening, dinnertime or PM administration,
and optionally each compartment has a foil or equivalent backing, or each compartment is an environmentally- (optionally moisture-, pathogen-, and/or light-) protected or sealed storage unit, and optionally the foil backing requires minimal finger strength to remove a dosage formulation (optionally one, two or three or more capsules, tablets, pills, geltabs or equivalents) in the compartment,
and optionally the blister package is a face seal blister package, a gang run blister package, a mock blister package, an interactive blister package or a slide blister package,
and optionally the drug delivery device or package, blister package, clamshell or tray is joined with board material which allows the product to be packaged, handled, hung, displayed and/or shipped without damaging the blister protection or seal, and optionally also provided with child resistant features,
and optionally the drug delivery device or package, blister package, clamshell or tray comprises a medical electronic monitory system that records administration time and transmits information to near-field communication (NFC) enabled mobile phone,
wherein optionally the kit is a travel kit comprising instructions of use by the traveler, wherein the instructions optionally comprise instructing the traveler to immediately take an indicated dosage, optionally a first arrange row of drugs on the drug delivery device or package, blister package, clamshell or tray, if they believe they have been in contact with or in near contact with or exposed to an individual that is infect or might be infected with coronavirus (for example, a COVID-19) or to an individual having fever, sore throat, rigors or shakes, chest pain on breathing, coughing, diarrhea and/or muscle aches.

In alternative embodiments, provided are methods for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the sub-family Orthocoronavirinae, or a virus in the family Coronaviridae, or a virus in the order Nidovirales, comprising administered to an individual in need thereof a therapeutic combination of drugs or drug, a pharmaceutical dosage form, a drug delivery device, or a product of manufacture as provided herein, wherein optionally the therapeutic combinations of drugs or drugs or pharmaceutical dosage forms are administered as inhaled or aerosol formulations (for example, powders, liquids, aerosols) and/or are administered (optionally simultaneously, or sequentially) with oral, intravenous (IV) or intramuscular formulations.

In alternative embodiments provided are products of manufacture fabricated or manufactured as an inhaler, asthma “puffer-like” device, nebulizer or nasal spray device or equivalent, for delivering a medication or a drug, as provided herein, for use in treating, preventing, ameliorating, slowing the progress of, decreasing the severity of or preventing a microbial infection in an individual in need thereof, wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus, and optionally the infection is caused by a virus in the subfamily Orthocoronavirinae, or a virus in the family Coronaviridae, or a virus in the order Nidovirales.

In alternative embodiments provided is a use of a product of manufacture fabricated or manufactured as an inhaler, asthma “puffer-like” device, nebulizer or nasal spray device or equivalent, for delivering a medication or a drug, as provided herein, in the manufacture of a medicament for treating, preventing, ameliorating, slowing the progress of, decreasing the severity of or preventing a microbial infection in an individual in need thereof, wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus, and optionally the infection is caused by a virus in the subfamily Orthocoronavirinae, or a virus in the family Coronaviridae, or a virus in the order Nidovirales.

In alternative embodiments, methods for treating, preventing, ameliorating, slowing the progress of, decreasing the severity of a coronavirus infection comprises administering a therapeutic combination of drugs or drug, a pharmaceutical dosage form, a drug delivery device, or a product of manufacture as provided herein to an individual that suffers from long term effects, or chronic effects or symptoms, of the viral infection, also called “long-haulers”, or people who have not fully recovered from COVID-19 weeks or even months after first experiencing symptoms, where some long haulers experience continuous symptoms for weeks or months, while others feel better for weeks, then relapse with old or new symptoms. In alternative embodiments, methods as provided herein are used to prevent the so-called “long-hauler” syndrome, or to treat or prevent continuous symptoms for weeks or months, or to prevent or treat relapsing with old or new symptoms.

In alternative embodiments, methods as provided herein further comprise use of an air ionizer (for example, a technical ionizers or a bioionizer), or a device capable of making or generating electrons and/or negatively-charged oxygen ions and/or positively-charged ions. In alternative embodiments, this air ionizer or device capable of generating electrons and/or ions is operatively connected to the product of manufacture (for example, medical device as provided herein, for example, respirator or ventilator, CPAP, an inhaler, asthma “puffer-like” device, nebulizer or nasal spray device or equivalent) such that the ionized air, or air comprising the electrons and/or ions, is delivered to a patient, optionally as a forced air or positive air pressure flow of air, to or into the nose and or mouth, and optionally to the trachea, bronchi or lungs, of a user. In alternative embodiments, this air ionizer or device capable of generating electrons and/or ions is a stand-alone device, and provides or delivers ionized air, or air comprising the electrons and/or ions, to a patient, optionally as a forced air or positive air pressure flow of air, to or into the nose and or mouth, and optionally delivers the ionized air, or air comprising the electrons and/or ions to the trachea, bronchi or lungs of a user. In alternative, embodiments, the air ionizer, or a device capable of making or generating electrons and/or negatively-charged oxygen ions and/or positively-charged ions, is connected to the inhalation port of a ventilator as provided herein, which is particularly therapeutic for COVID-19 patients.

In alternative embodiments, this air ionizer or device capable of generating electrons and/or ions is used with or without, or optionally further comprises, a humidifier. In alternative embodiments, the ionizer and/or the humidifier is a stand-alone device, or is part of or is configured with or operatively connected to a product of manufacture as provided herein, or is part of or is configured with or operatively connected to a stand alone air ionizer or device capable of generating electrons and/or ions.

In alternative embodiments, the air ionizer or device capable of generating electrons and/or ions, and/or the humidifier, is placed or configured near the patient or person requiring prevention of the condition, for example, the air ionizer or device capable of generating electrons and/or ions, and/or the humidifier, is placed close to the nose, or is suspended from the neck, or is placed on a table or stand near the patient, or positioned on a bed or chair, for example, positioned between about 0.5 to 3 or between about I to 2 meters away from the patient or patient's face. In alternative embodiments, the air ionizer or device capable of generating electrons and/or ions, and/or the humidifier, is run independently of a product of manufacture as provided herein (for example, an inhaler, asthma “puffer-like” device, nebulizer or nasal spray device, a respirator or ventilator, a warm or hot air delivery device, and/or a CPAP device), for example, can be run continuously, even in sleep. In this way the air ionizer or device capable of generating electrons and/or ions does not need to be connected to any other product, including a product of manufacture as provided herein, and optionally can be run or controlled independently of the therapeutic administration of drugs as provided herein.

In alternative embodiments, provided are methods for treating, preventing, ameliorating, slowing the progress of, decreasing the severity of or preventing a microbial infection in an individual in need thereof comprising administering a medication or a drug combination as contained in a product of manufacture fabricated or manufactured as an inhaler, asthma “puffer-like” device, nebulizer or nasal spray device or equivalent, for delivering a medication or a drug, as provided herein,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

In alternative embodiments of methods as provided herein, the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a COVID-19 infection.

In alternative embodiments, provided are uses of a product of manufacture fabricated or manufactured as an inhaler, asthma “puffer-like” device, nebulizer or nasal spray device or equivalent, as provided herein, for delivering a medication or a drug,
wherein optionally the delivering of the medication or the drug is for treating, preventing, ameliorating, slowing the progress of, decreasing the severity of or preventing a microbial infection in an individual in need thereof comprising administering a medication or a drug combination,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

In alternative embodiments, provided are products of manufacture fabricated or manufactured as an inhaler, asthma “puffer-like” device, nebulizer or nasal spray device or equivalent, as provided herein, for use in delivering a medication or a drug,
wherein optionally the delivering of the medication or the drug is for treating, preventing, ameliorating, slowing the progress of, decreasing the severity of or preventing a microbial infection in an individual in need thereof comprising administering a medication or a drug combination,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

The details of one or more exemplary embodiments of the invention are set forth in the description below. Other features, objects, and advantages of the invention will be apparent from the description and from the claims.

All publications, patents, patent applications cited herein are hereby expressly incorporated by reference in their entireties for all purposes.

### DESCRIPTION OF EMBODIMENTS

In alternative embodiments, provided are products of manufacture fabricated or manufactured as medical devices such as an inhaler, asthma “puffer-like” device, nebulizer or nasal spray device, a respirator or ventilator, a warm or hot air delivery device (such as a modified hairdryer), and/or a CPAP (continuous positive air pressure) device (for example, for managing or treating sleep apnea) or equivalent, for delivering one or a combination of medications or drugs, and/or for delivering ionized air or air comprising generated electrons and/or negatively-charged oxygen ions and/or positively-charged ions, and/or for delivering warm or hot air. In alternative embodiments, provided are combinations of different medications, and new devices for administering them, which are used together can treat, ameliorate, slow the progress of, decrease the severity of or prevent: a coronavirus infection such as a 2019-nCoV infection, or SARS (Severe Acute Respiratory Syndrome) or MERS (Middle East Respiratory Syndrome), or any respiratory infection such as an influenza virus, respiratory syncytial virus, picornavirus, parainfluenza virus, adenovirus, rhinovirus, human metapneumovirus, hantaviruses or enterovirus, or any other respiratory conditions such as sinusitis, asthma, bronchiectasis, bronchitis, chronic bronchitis, chronic obstructive airways disease, pulmonary fibrosis, bacterial pneumonia of any bacterial, mycobacterial and fungal origin, sarcoidosis, vasculitis and/or a granulomatous disease. In alternative embodiments, provided are novel methods of administration dosing to cover the period of exposure, diagnosis, and treatment.

In alternative embodiments, products of manufacture such as medical devices, for example, inhalers and nebulizers, compositions, including preparations, formulations and/or kits, comprise or deliver combinations of ingredients, for example, therapeutic combinations as described herein, by inhalation of liquids, powders and/or mists.

In alternative embodiments, therapeutic combinations and formulations drug combination, or pharmaceutical preparations or pharmaceutical compositions delivered by products of manufacture as provided herein are formulated, for example, as a powder, for example, as lyophilized material, for example, a lyophilized encapsulated product.

In alternative embodiments, products of manufacture as provided herein are drug delivery devices such as respirators or ventilators, inhalers, nebulizers, nasal sprays and equivalents (for example, an asthma “puffer-like” device), including for example, portable inhalation devices, inhalers, nebulizers or nasal spray devices, warm or hot air delivery devices (such as a modified hairdryer), and CPAP (continuous positive air pressure) devices (for example, for managing or treating sleep apnea), for the delivery of medications or drugs, including delivery of a therapeutic combination of drugs, a pharmaceutical dosage form or a formulation as provided herein.

In alternative embodiments, a drug delivery device as provided herein is or is fabricated or manufactured as an inhaler, asthma “puffer-like” device, nebulizer or nasal spray device or equivalent, and optionally is a hand-held or otherwise portable device, and optionally the device is a metered or dose-counting inhaler or a nasal spray device.

In alternative embodiments, a drug delivery device as provided herein, for example, the inhaler, asthma “puffer-like” device, nebulizer, or the nasal spray device is, or comprises parts of, or is made or used, by a method or a device as described in for example, U.S. Pat. No. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol or aerosol imparted with flavor, a sensor configured to detect a predefined variable, an interface configured to make a notification to an inhaler of the aerosol, and a controller), or U.S. Pat. No. 10,463,815 (describing a dry powder inhaler may include a powder storage region, an inlet channel, a dispersion chamber, and an outlet channel); or U.S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol and/or a vapour in an inhaler device includes a reservoir for storing a supply of a liquid; a heating system fluidly connected with the reservoir for receiving the liquid and configured to heat the liquid to generate the aerosol and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, for example, as described in U.S. Pat. No. 10,561,808.

In alternative embodiments, a medical device as provided herein, for example, a respirator or ventilator, a nebulizer, an inhaler or a nasal spray device, or the like, is a hand-held or otherwise portable device, and can be used or is intended for use in any public space, for example, in any vehicle or mode of transport, for example, on public transport such as buses, trams, trains, aircraft and/or boats, or in places of commerce such as stores, bars, sporting events, movies theaters, theater, musical events, or any gathering of people. In alternative embodiments, a medical device as provided herein, for example, a CPAP device, a respirator or ventilator, a nebulizer, an inhaler, a nasal spray device, a hot air delivery device (such as a modified hairdryer) as provided herein, and the like, comprises a cassette, packette, interchangeable disk (for example, for holding a powder or mixture of powders) or reservoir (optionally a refillable reservoir) in or on the product of manufacture, or a removable cassette or packette, interchangeable disk (for example, for holding a powder) that can be inserted into a slot or port on the product of manufacture, or a separate reservoir or container operatively linked or joined to the product of manufacture, that comprises a drug or a medication, for example, a combination of drugs or a formulation as provided herein, for delivery of the drug or medication to a user of the medical device. In alternative embodiments, a defined amount of drug or medication is administered each time the device, for example, inhaler, nebulizer, is activated by the user, and the drug or medication is thus effectively carried into the bronchi and lungs of the used by inhalation. In alternative embodiments, for example as with a respirator or ventilator, the device comprises a timing device such that a predefined or preset amount of drug or medication is administered at predefined or set times or intervals.

In alternative embodiments, the medical device as provided herein administers by inhalation, or by forced air injected into the bronchi and/or lungs (for example, hot forced air), a drug or medication, for example, a formulation or combination of drugs as provided herein. For example, the inhaled or aerosol material (for example, powder or liquid, for example, as a mist or water droplets) can comprise one or drugs, formulations or agents to fight infection, prevent colonization of the entire respiratory mucosa including alveolei and/or lungs by an infectious agent, or to allow an immunization (to administer a vaccine), or module an immune response (for example, comprise an immunomodulator such as an immunosuppressive drug such as tocilizumab or atlizumab, or ACTEMRA™, ROACTEMRA™). In alternative embodiments, the medical device delivers a biofilm dissolving agent (dornase alfa (for example, PULMOZYNE™)), an anticholinergic (optionally tiotropium bromide or SPIRIVA™), a bronchodilator, a corticosteroid, an antileukotriene, or an asthma drug (optionally formoterol, salbutamol, albuterol, or VENTOLIN™). For example, to treat or prevent an infection by the COVID-19 virus, the device (for example, nebulizer, inhaler) can comprises a liquid or a powdered product comprising for example, chloroquine, hydroxychloroquine or derivatives of chloroquine; or, zanamivir (for example, RELENZA™), oseltamivir (or TAMIFLU™); ritonavir, darunavir, nelfinavir (for example, VIRACEPT™), amprenavir (for example, AGENERASE™), favilavir and/or remdesivir (for example, GS-5734™, Gilead Sciences), and optionally also comprising an antibiotic, for example, azithromycin (for example, ZITHROMAX™, or AZITHROCIN™, optionally an oral extended-release formulation of azithromycin, or ZMAX™); and/or other antiviral or antimicrobial agents, any of which or all can be inhaled simultaneously, or sequentially, to develop a high concentration of the antiviral or antimicrobial agents in the respiratory tract.

### Hand-Held or Portable Devices

In alternative embodiments, a portable, for example, hand-held (or worn around the neck), medical devices, for example, inhaler, ionizer, asthma puffer or nebulizer, as provided herein administers an inhalation product (for example, powder, mist, any liquid spray) that can comprise various classes of medications, such as antivirals that focus on or target (for example, for treatment, prevention or amelioration) a respiratory virus such as influenza, a cold virus and/or a coronavirus, and can also comprise an immunomodulator, for example, immune stimulant, or an immunosuppressive drug such as tocilizumab or atlizumab, or ACTEMRA™, ROACTEMRA™, and agents specifically to give people who are flying on an aircraft, travelling in boats, trains, buses and taxis and the like, an ability to any time (for example, at defined times) spray using a device as provided herein to spray or administer the drug or medication to the back of a throat, wherein the anti-infective agent can prevent the user from catching most infections by inhalation. In alternative embodiments, the portable, for example, hand-held, medical devices, for example, inhalers or nebulizers, as provided herein can be purchased from various dispensaries or stores, such as pharmacies, on aircraft, airports, on buses and in cabs so that a person who may have forgotten to obtain the product can then acquire it and take it on their overseas trips and holidays.

In alternative embodiments, a portable, for example, hand-held, medical devices, for example, inhaler, asthma puffer or nebulizer, as provided herein can administer ionized air or air comprising generated electrons and/or negatively-charged oxygen ions and/or positively-charged ions, as discussed below.

As discussed above, in alternative embodiments, the portable or hand-held medical device comprises a cassette, packette, interchangeable disk (for example, for holding a powder) or reservoir (optionally a refillable reservoir) in or on the product of manufacture, or a removable cassette or packette, interchangeable disk (for example, for holding a powder) that can be inserted into a slot or port on the product of manufacture, or a separate reservoir or container operatively linked or joined to the product of manufacture, that comprises a drug or a medication for inhalation delivery of the drug or medication to a user. In alternative embodiments, a modified hairdryer-type medical device as provided herein provides an adjustable temperature, adjustable air intake; a provision (or receptacle) for insertion of a drug-containing cassette (optionally providing or delivering a combination of medications); and/or warm-to hot air availability (optionally with temperature control) to inhibit viral and bacterial growth, as discussed in further detail below.

In alternative embodiments, a medical device as provided herein for inhalation delivery of a drug or a medication or combinations thereof to a user is fabricated as a meter-dose inhaler (MDI) (either open or closed mouth MDI), which can comprise a pressurized canister of the drug or medication in a plastic case with a mouthpiece, and a holding chamber having a plastic tube with a mouthpiece, a valve to control mist delivery and a soft sealed end to hold the MDI; the holding chamber can assist delivery of the drug or medication to the nose and/or lungs, for example, as an AEROCHAMBER™ device. In alternative embodiments, the inhaler or nebulizer is breath activated, for example, as an REDIHALER™ device.

In alternative embodiments, a medical device as provided herein for inhalation delivery of a drug or a medication or combinations thereof to a user is fabricated a dry powder inhaler (such as a dry powder disk inhaler, for example, as a DISKUS™ device), optionally having a dose counter window so user can see how many doses are left), for example, where the powder is dose dispensed by (using) a disposable, refillable or replaceable cassette, packette or disk; and the dry powder dispensing can be breath activated, for example, as an AEROLIZER™, FLEXHALER™, PRESSAIR™, DISKUS™, HANDIHALER™, TWISTHALER™, ELLIPTA™, NEOHALER™, RESPICLICK™, ROTAHALER™ or TUBUHALER™ device.

In alternative embodiments, a medical device as provided herein for inhalation delivery of a drug or a medication or combinations thereof to a user is fabricated a nebulizer or soft mist inhaler, which can comprise a nebulizer delivery system comprising a nebulizer (for example, a small plastic bowl with a screw-top lid) and a source for compressed air to generate a mist comprising the drug or medication, which also can be dose dispensed using a disposable, refillable or replaceable cassette, packette or disk.

### Hot Air or Forced Air Delivery Device

In alternative embodiments, medical devices as provided herein, including respirators or ventilators, CPAP devices, and portable, for example, hand-held, medical devices, for example, an inhaler, asthma puffer or nebulizer as provided herein, is capable of delivering forced air and/or hot air, or is capable of administering ionized air or air comprising generated electrons and/or negatively-charged oxygen ions and/or positively-charged ions, and can comprise a mechanism analogous to a hair dryer or so-called blow dryer, where warm to hot air, steam or vapor or just room temperature air, is forcefully delivered directly into the user's mouth and/or nose, thereby having the forced air, and/or the hot or warm air, permeate through the nose into the sinuses and turbinates, down the trachea, and into the main bronchi and bronchioles. The device can have controls such as dials to set or adjust the warmth or heat of the air and/or, to adjust the force the air is delivered into the nose or mouth, and bronchi and lungs. The device can be battery powered. The device can comprise (optionally a disposable) mouthpiece (optionally a two-way mouthpiece) to fit or place the device over the mouth for air delivery, or a face piece to fit over both the nose and the mouth for simultaneous air delivery to both nose and mouth, and optionally permitting inhaling and exhaling when a side valve opens. For example, the medical device can comprise components to facilitate the generation of warm or hot air, the delivery of the warm or hot air, and/or adjusting the heat or force of air delivery, as described for example, in U.S. Pat. Nos. 10,492,585; 10,485,320; 10,405,630 (describing a battery powered blow dryer having a heating element powered by an attached battery pack); U.S. Pat. Nos. 10,299,560; 10,299,559; and/or 10,143,284. The device can also comprise a noise cancelation device, for example, as in U.S. Pat. No. 10,089,973.

As discussed above, in alternative embodiments, the medical device delivers drugs or medications from a renewable or disposable or refillable cassettes, packettes, interchangeable disk (for example, for holding a powder) or drug-comprising modules disposed onto or in the medical device. In alternative embodiments, the medical device comprises an adjustable value or any mechanism that modulates or regulates the amount of the medication or the drug delivered to the to or into the path of the airflow and therefore the amount of drug or medication that pass into the nose, trachea, bronchi and/or lungs. In alternative embodiments, asthma or anti-microbial (for example, antiviral) drugs can be so administered, for example an antibiotic agent or drug (such as gentamycin), a biofilm dissolving agent, an anticholinergic (optionally tiotropium bromide or SPIRIVA™), a bronchodilator, steroid, a corticosteroid, a antileukotriene, or an asthma drug (optionally formoterol, salbutamol, albuterol, or VENTOLIN™) or any formulation or drug combination as provided herein, can be so administered.

Antifungal agents can be used if someone is going overseas and they get recurrent urine infections or sinus or vaginal fungal infections, for example, itraconazole (for example, SPORANOX™, SPORAZ™, ORUNGAL™) can be administered. Medications can be administered with/without anti-inflammatory agents appropriate to that patient's recurrent infections or other illnesses. These antifungals also can help treat (and optionally cure) chronic and recurrent sinusitis and asthma, and the antifungals can be combined with a specific antibiotic for an infection of the sinus, and can comprise lincomycin, gentamycin and others in this group, dexamethasone, ansamycins for example rifampicin, rifabutin, and other rifamycins. Such combinations can be used to manage relapse of asthma, bronchitis, bronchiectasis, and chronic obstructive airways disease.

In alternative embodiments, a medical device as provided herein for inhalation delivery of a drug or a medication or combinations thereof to a user is fabricated a dry powder inhaler (such as a dry powder disk inhaler, for example, as a DISKUS™ device), optionally having a dose counter window so user can see how many doses are left), for example, where the powder is dose dispensed by (using) a disposable, refillable or replaceable cassette, packette or disk; and the dry powder dispensing can be breath activated, for example, as an AEROLIZER™, FLEXHALER™, PRESSAIR™, DISKUS™, HANDIHALER™, TWISTHALER™, ELLIPTA™, NEOHALER™, RESPICLICK™, ROTAHALER™ or TUBUHALER™ device.

### CPAP (Continuous Positive Air Pressure) Devices

In alternative embodiments, provided are CPAP (continuous positive air pressure) devices, for example, as used in sleep apnea treatments, to deliver a drug or medication, for example, a drug or formulation as provided herein, or for the administration of ionized air or air comprising generated electrons and/or negatively-charged oxygen ions and/or positively-charged ions. In alternative embodiments, when a user is inhaling during sleep, for example, through an apnoea phase during sleep, a therapeutic agent intermittently delivered, for example, by a drug or medication-comprising cassette or packette, interchangeable disk (for example, for holding a powder), as discussed above. For example, as a patient is breathing through the CPAP, which can be for many hours, the device can be set for intermittent delivery or administration of medication or drugs and/or forced warm or hot air, and/or humidified air (for example, as described in U.S. Pat. No. 10,518,061, describing a humidifier for humidification of air to be delivered to a patient's airways), during the night to fight an infection, for example, a throat or a chest infection.

In alternative embodiments, asthma or anti-microbial (for example, antiviral) drugs can be administered via the CPAP, for example an antibiotic agent or drug (such as gentamycin), a biofilm dissolving agent (optionally PULMOZYME™), an anticholinergic (optionally tiotropium bromide or SPIRIVA™), a bronchodilator, steroid, a corticosteroid, a antileukotriene, or an asthma drug (optionally formoterol, salbutamol, albuterol, or VENTOLIN™) or any formulation or drug combination as provided herein, can be administered using the CPAP. Other drugs that can be administered include steroids for asthma and systemic antibiotics such as gentamicin or vancomycin; optionally in this embodiment a CPAP is used to treat urine infections by administration of an antibiotic (for example, an antibacterial or an antifungal) such as gentamycin, which is excreted through the urinary tract.

In alternative embodiments, CPAP devices as provided herein can comprise components or be fabricated as, or be used, as described in for example, U.S. Pat. Nos. 10,595,814; 10,549,057 (describing a ventilator system includes a mask to be placed over a wearer's face); 10,543,333 (describing a vent arrangement for a mask or associated conduit to discharge exhaled gas from the mask); and/or 10,406,312 (describing a CPAP flow driver for using nebulizer with CPAP apparatus).

In alternative embodiments, a medical device as provided herein for inhalation delivery of a drug or a medication or combinations thereof to a user is fabricated a dry powder inhaler (such as a dry powder disk inhaler, for example, as a DISKUS™ device), optionally having a dose counter window so user can see how many doses are left), for example, where the powder is dose dispensed by (using) a disposable, refillable or replaceable cassette, packette or disk; and the dry powder dispensing can be breath activated, for example, as an AEROLIZER™, FLEXHALER™, PRESSAIR™, DISKUS™, HANDIHALER™, TWISTHALER™, ELLIPTA™, NEOHALER™, RESPICLICK™, ROTAHALER™ or TUBUHALER™ device.

### Respirators or Ventilators

In alternative embodiments, provided are respirators or ventilators comprising components as described herein, including respirators or ventilators for the administration of forced air or forced hot or warm air, or for the administration of ionized air or air comprising generated electrons and/or negatively-charged oxygen ions and/or positively-charged ions, or for the controlled administration of drugs or medications, including formulations and drug combinations as provided herein, optionally by use of a removable and/or disposable drug-comprising module, cassette or packette or interchangeable disk (for example, for holding a powder), as described above.

In alternative embodiments, respirators or ventilators as provided are used on patients in intensive care; for example, as during the coronavirus (COVID-19) infection epidemic. In alternative embodiments, respirators or ventilators as provided have various attachments including a nebulizing attachment or equivalent, and in the nebulizer a drug or medication can be added, including for example, an antiviral formulation or drug combination as provided herein, or an antibiotic agent or drug (such as gentamycin), a biofilm dissolving agent (optionally PULMOZYME™), an anticholinergic (optionally tiotropium bromide or SPIRIVA™), a bronchodilator, steroid, a corticosteroid, a antileukotriene, or an asthma drug (optionally formoterol, salbutamol, albuterol, or VENTOLIN™) or any formulation or drug combination as provided herein.

In alternative embodiments, the temperature and humidity of air delivered by the respirator or ventilator is adjusted and/or modified, for example, by having a special in-series tube inserted which can warm or heat an air to a pre-determined temperature, for example, to eradicate, kill or at least ameliorate the effects of a respiratory virus, for example, coronavirus such as COVID-19. In alternative embodiments, the temperature of the air delivered by the device is adjusted or is adjustable to above about 53° C., or to between about 60° C. to 70° C., or 55° C. to 90° C., which can kill a coronavirus. Inspired temperatures in the range of between about 55° C. to 90° C. or more, or about 60° C. to 70° C. can slowly be reached by increasing the heat of the infused or inhaled air, steam, mist or vapor until the viral infectious agent is killed because of the heat. In alternative embodiments, this heat treatment is also be used for any pneumonia or asthma, and can include a biofilm dissolving agents such as dornase alfa (for example, PULMOZYME™). In alternative embodiments, a device as provided herein comprises a temperature-sensing device at the lung (or distal) end of an endotracheal tube to monitor the heat delivered by the device, for example, a respirator or ventilator as provided herein.

In alternative embodiments, a medical device as provided herein for inhalation delivery of a drug or a medication or combinations thereof to a user is fabricated a dry powder inhaler (such as a dry powder disk inhaler, for example, as a DISKUS™ device), optionally having a dose counter window so user can see how many doses are left), for example, where the powder is dose dispensed by (using) a disposable, refillable or replaceable cassette, packette or disk; and the dry powder dispensing can be breath activated, for example, as an AEROLIZER™, FLEXHALER™, PRESSAIR™, DISKUS™, HANDIHALER™, TWISTHALER™, ELLIPTA™, NEOHALER™, RESPICLICK™, ROTAHALER™ or TUBUHALER™ device.

### Filtering and Irradiation

In alternative embodiments, devices as provided herein further comprise, or are fitted with, air purification or filtering systems for removing airborne particles such as bacterial or viral particles, or ionizers or devices capable of generating electrons and/or negatively-charged oxygen ions and/or positively-charged ions. In alternative embodiments, devices as provided herein further comprise, or are fitted with, ultraviolet light or other irradiation mechanisms to kill or inactivate microbes, thus ensuring particle and/or infection-free air delivery.

### Ionizers

In alternative embodiments, products of manufacture as provided herein, including medical devices such as an inhaler, asthma “puffer-like” device, nebulizer or nasal spray device, a respirator or ventilator as provided herein, further comprise an air ionizer (for example, a technical ionizer or a bioionizer) or a device capable of making or generating electrons and/or negatively-charged oxygen ions and/or positively-charged ions, where this air ionizer or device is capable of generating electrons and/or ions, and is operatively connected to or built or configured into the product of manufacture such that the ionized air, or air comprising the electrons and/or ions, is delivered to a patient, optionally as a forced air or positive air pressure flow of air, to or into the nose and or mouth, and optionally to the trachea, bronchi or lungs, of a user.

In alternative embodiments, products of manufacture as provided herein are operatively linked to an air ionizer or a device capable of making or generating electrons and/or negatively-charged oxygen ions and/or positively-charged ions, where this air ionizer or device is capable of generating electrons and/or ions, such that the air ionizer or the device is operatively connected to a product of manufacture as provided herein such that the ionized air, or air comprising the electrons and/or ions, is delivered to a patient, optionally as a forced air or positive air pressure flow of air, to or into the nose and or mouth, and optionally to the trachea, bronchi or lungs, of a user. In alternative embodiments, the air ionizer or device is capable of generating electrons and/or ions is: an AirClean E7™ HEPA air purifier, a AVICHE™ or WOOLALA™ Personal Air Purifier, an Elanra Medical IonMax™, ION90™, Life MXL-15™ (Lifeionizers), or a Lustre™ Ionizer.

In alternative embodiments, while the invention is not limited by any particular mechanism of action, the positively and/or negatively charged particles when inhaled or otherwise forced into the nose and or mouth, and optionally to the trachea, bronchi or lungs, of a user, affect (for example, kill or otherwise inhibit the ability of the virus to multiply, or negatively affect the virulency of the virus) an inhaled coronavirus and/or other inspired virus (for example, such as influenza, rhinovirus, picornavirus, adenovirus or enterovirus), and/or viruses multiplying in the respiratory tract.

In alternative embodiments, a product of manufacture as provided herein, including a product of manufacture comprising an air ionizer or a device capable of making or generating electrons and/or negatively-charged oxygen ions and/or positively-charged ions, or a product of manufacture operatively connected to such a device, or a product of manufacture optionally also comprising or operatively connected to a humidifier and/or source of warm air, is used in the treatment and/or prevention of: a coronavirus infection such as a 2019-nCoV infection, or SARS (Severe Acute Respiratory Syndrome) or MERS (Middle East Respiratory Syndrome), or any respiratory infection such as an influenza virus, respiratory syncytial virus, picornavirus, parainfluenza virus, adenovirus, rhinovirus, human metapneumovirus, hantaviruses or enterovirus, or any other respiratory conditions such as sinusitis, asthma, bronchiectasis, bronchitis, chronic bronchitis, chronic obstructive airways disease, pulmonary fibrosis, bacterial pneumonia of any bacterial, mycobacterial and fungal origin, sarcoidosis, vasculitis and/or a granulomatous disease, or asthma, pneumonia, bronchiectasis, tuberculosis (TB) and/or acute and chronic bronchitis.

### Formulations

In alternative embodiments, any drug or therapeutic, including a chloroquine (for example, ARALEN™), chloroquine phosphate, quinine, chloroquine diphosphate, hydroxychloroquine (for example, PLAQUENIL™), lopinavir, ritonavir, azithromycin (for example, ZITHROMAX™, or AZITHROCIN™, optionally an oral extended-release formulation of azithromycin, or ZMAX™), opaganib or YELIVA™, and/or oseltamivir, and/or an anti-viral drug or drug combination or medication as provided herein, or an anti-microbial drug as provided herein, is formulated as a liquid, powder (for example, a dry powder), a microparticle or a nanoparticle, spray or an aerosol. In alternative embodiments, the powder can be an agglomeration of powder particles or an agglomerate having irregular geometries such as width, diameter, and length. In alternative embodiments, the dry powder can be formulated as a granule of a physiologically acceptable excipient to be used as a carrier for a dry powder formulation for inhalation as described for example, in U.S. Pat. No. 10,583,085. In alternative embodiments, other agents such as favipiravir, ivermectin, moxidectin, doramectin, selamectin, a milbemycin (optionally milbemectin, milbemycin oxime, moxidectin or nemadectin, remdesivir, aviptadil, and/or camostat are administered as inhalations (for example, powders), and any or all also can be administered orally or by other routes, for example, remdesivir can be administered intravenously (IV). Interferon and hydroxychloroquine also can be administered by inhalation.

In alternative embodiments, hyper-immune plasma from a recuperating patient is administered to speed up recovery by IV infusion.

In alternative embodiments, the chloroquine (for example, ARALEN™), chloroquine phosphate, quinine, chloroquine diphosphate, hydroxychloroquine (for example, PLAQUENIL™) is formulated at between about 50% to 100% concentration as a liquid or aqueous formulation. In alternative embodiments, the chloroquine (for example, ARALEN™), chloroquine phosphate, quinine, chloroquine diphosphate, hydroxychloroquine (for example, PLAQUENIL™) is formulated and delivered at a dosage regimen of about 0.2 mg/kg to about 150 mg/kg per dose.

In alternative embodiments, the chloroquine (for example, ARALEN™), chloroquine phosphate, quinine, chloroquine diphosphate, hydroxychloroquine (for example, PLAQUENIL™) is combined with, or administered with, azithromycin (for example, ZITHROMAX™, or AZITHROCIN™), and optionally the chloroquine, chloroquine phosphate, quinine, chloroquine diphosphate, hydroxychloroquine and/or azithromycin are formulated as slow release formulations, for example, formulated in microspheres as oral extended-release formulations, for example, azithromycin in the form of ZMAX™.

In alternative embodiments, the invention provides pharmaceutical formulations or compositions for use in in vivo, in vitro or ex vivo methods to treat, prevent, reverse and/or ameliorate a viral infection, for example, coronavirus (optionally COVID-19).

In alternative embodiments, the pharmaceutical compositions as provided herein or used to practice methods as provided herein can be administered parenterally, topically, orally or by local administration, such as by aerosol or transdermally. These pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”). For example, in alternative embodiments, these compositions of the invention are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods as provided herein can be in forms such as tablets, pills, powders, capsules, liquids, gels, syrups, slurries, suspensions, etc., suitable for in vivo, in vitro or ex vivo applications.

In alternative embodiment, compounds (for example, formulations) as provided herein or used to practice methods as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations as provided herein or used to practice methods as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

In alternative embodiments, provided are products of manufacture for delivering compositions and formulations as provided herein or used to practice methods as provided herein using liposomes or nanoparticles. By using liposomes or nanoparticles, particularly where the liposome or nanoparticle surfaces carry ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

### Nanoparticles, Nanolipoparticles and Liposomes

In alternative embodiments, provided are products of manufacture for delivering nanoparticles, nanolipoparticles, vesicles and liposomal membranes comprising compounds or mixtures of compounds as provided herein or used to practice methods as provided herein. For example, in alternative embodiments, an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™) is formulated and/or administered in a liposome or nanoparticle formulation.

In alternative embodiments, provided are multilayered liposomes comprising compounds or mixtures of compounds used to practice methods as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, for example, as described in Park, et al., U.S. Pat. Pub. No. 20070042031, including method of producing a liposome by encapsulating an active agent, the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, for example, for targeting delivery of a compound, as described for example, in U.S. Pat. Pub. No. 20070110798.

The invention also provides nanoparticles comprising compounds used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions used to practice methods as provided herein to mammalian cells in vivo, in vitro or ex vivo, as described, for example, in U.S. Pat. Pub. No. 20050136121.

### Methods of Administration

In alternative embodiments, methods of delivery of the chloroquine (for example, ARALEN™), chloroquine phosphate, quinine, chloroquine diphosphate, hydroxychloroquine (for example, PLAQUENIL™), lopinavir, ritonavir and/or oseltamivir, and/or the anti-viral drug or medication, or anti-microbial drug, comprise treatment regimens where the drug, medication or combination of drugs are administered every hour, every other hour, once, twice, three, four, five, six, seven, eight, nine, ten, eleven or twelve times a day. In alternative embodiments, the length of time of treatment, or the exact dosaging or dosage regimen, is determined by the clinician, or the administration is to begin immediately after possible exposure to an individual having (or exposed to another individual having) a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, the chloroquine (for example, ARALEN™), chloroquine phosphate, quinine, chloroquine diphosphate, hydroxychloroquine (for example, PLAQUENIL™) is formulated at between about 50% to 100% concentration as a liquid or aqueous formulation, which can be used either as an aerosol and/or given orally. In alternative embodiments, the chloroquine (for example, ARALEN™), chloroquine phosphate, quinine, chloroquine diphosphate, hydroxychloroquine (for example, PLAQUENIL™) is formulated and delivered (for example, by inhalation and/or orally) at a dosage regimen of about 0.2 mg/kg to about 150 mg/kg per dose.

### Dosaging for Therapeutic or Prophylactic Purposes

In alternative embodiments, provided are drug combinations and drug delivery devices comprising these combinations for therapeutic and/or prophylactic (prevention) purposes.

In alternative embodiments, a therapeutic or a prophylactic drug or ingredient combination “package”, which can be a nebulizer, inhaler, respirator or CPAP insert, or the like, is designed such that a particular drug or ingredient combination (for example, a drug or ingredient combination having 2, 3, 4, 5, or 6 ingredients or active agents, wherein one, several or all are separately formulated or formulated into one delivery agent such as a capsule or geltab, or nebulizer, inhaler, respirator or CPAP insert), to be taken by a user every day, every other day, every week, every two weeks or every 4 weeks (i.e., monthly). In alternative embodiments, the therapeutic or a prophylactic drug combination “package” is designed (for example, instructing the user) to take the drug combination as a staggered dosage, for example, one administration of the drug combination for two or three days in a row staggered by a week before the next two or three day administration cycle begins again.

In alternative embodiments, the therapeutic or prophylactic drug or ingredient combination comprises:
(1)(a) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 10 mg to 80 mg dosages, or 12 to 60 mg dosages; and (b) chloroquine (optionally, ARALEN™), chloroquine phosphate, chloroquine diphosphate or hydroxychloroquine (optionally, PLAQUENIL™);
(2) the combination of (1)(a) and (1)(b) also with vitamin D, vitamin D2 (or ergocalciferol), vitamin D3 (or cholecalciferol) optionally at a dosage of between about 10,000 to 100,000 units per day;
(3) the combination of (1)(a) and (1)(b) also with zinc (optionally a zinc sulphate, acetate, gluconate or picolinate) optionally at a dosage of between about 1 mg to 250 mg;
(4) the combination of (2)(a) and (2)(b) also with zinc (optionally a zinc sulphate, acetate, gluconate or picolinate) optionally at a dosage of between about 1 mg to 250 mg;
(5) the combination of (1)(a) and (1)(b) also with a tetracycline class drug, wherein optionally the tetracycline class drug comprises doxycycline, or DORYX™, DOXYHEXA™, DOXYLIN™, optionally dosages at between about 100 mg to 600 mg per day, optionally between about 200 mg to 400 mg;
(6) the combination of (2)(a) and (2)(b) also with a tetracycline class drug, wherein optionally the tetracycline class drug comprises doxycycline, or DORYX™, DOXYHEXA™, DOXYLIN™, optionally dosages at between about 100 mg to 600 mg per day, optionally between about 200 mg to 400 mg;
(7) the combination of (4) also with a tetracycline class drug, wherein optionally the tetracycline class drug comprises doxycycline, or DORYX™, DOXYHEXA™, DOXYLIN™, optionally dosages at between about 100 mg to 600 mg per day, optionally between about 200 mg to 400 mg.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

These examples demonstrate that methods and products of manufacture as provided are effective for treating, preventing, ameliorating, slowing the progress of, decreasing the severity of or preventing a coronavirus infection, or an infection caused by a virus in the subfamily Orthocoronavirinae, or a virus in the family Coronaviridae, or a virus in the order Nidovirales.

### Example 1: Exemplary Treatment Regimens

A 29-year-old tourist is returning to Australia after a trip to northern Italy feels he may have acquired a respiratory infection and even COVID-19. He is given an exemplary specialized, preventative inhaler as provided herein on the plane home, which contains (or administers) powdered hydroxychloroquine and remdesivir, combined in the inhalation cassette or disc with zanamivir (RELENZA™). He is required to inhale three times over the 18 hour (hr) flight. Over the next 6 hours (hrs) his sore throat, cough and muscle pains disappear. He can breathe more easily. Given his symptoms he feels that he has a typical flu or early coronavirus infection. On arrival in Australia he is given a nasopharyngeal swab, which shows no evidence of any coronavirus infection. But a week later his serum test comes up positive for IgM antibody for coronavirus. This indicates the inhalant can ward off an early coronavirus chest infection, but the mucosal exposure still shows antibodies present, hence immunity.

### Example 2

A 48-year-old businessman travelling to the United States from Sydney develops a sore throat upon arrival at Dallas Fort Worth. Upon arrival at his hotel he is able to use an exemplary adjustable hairdryer as provided herein into which he inserts a disc or cassette containing multiple anti-viral powders, wherein optionally the disc or cassette comprises powders of: remdesivir (for example, GS-5734™, Gilead Sciences) and oseltamivir (for example, TAMIFLU™); chloroquine (or ARALEN™), chloroquine phosphate, quinine, chloroquine diphosphate and/or hydroxychloroquine (for example, PLAQUENIL™) with remdesivir and/or oseltamivir; remdesivir and/or oseltamivir remdesivir and/or oseltamivir, and opaganib or YELIVA™; opaganib and chloroquine (or ARALEN™), chloroquine phosphate, quinine, chloroquine diphosphate and/or hydroxychloroquine (for example, PLAQUENIL™); or, opaganib, hydroxychloroquine and azithromycin (for example, ZITHROMAX™, or AZITHROCIN™, optionally an extended-release formulation of azithromycin, or ZMAX™). Upon inhaling the powders, which can take about 10 minutes, he continues to inhale air at a temperature of about 65° C., slowly adjusting temperature upwards from around 50° C. He waits for another two hours and repeats. By six hours his throat improves and he is able to function normally from then on.

Even though he does not undergo an initial nasal swab for coronavirus, several weeks later he does have serology which shows the presence of both IgM and IgG for COVID-19. This indicates exposure to coronavirus without manifestation of full symptoms from the coronavirus infection. Use of the hairdryer that included heated air and anti-viral agents treats the infection and prevents manifestation of full symptoms.

### Example 3

A 65 year old female is admitted to hospital having difficulties breathing. She is immediately put on an exemplary respirator or ventilator as provided herein, and the operator inserts a cassette or a disc containing anti-viral powders (as used or provided for in Example 2) in a receiving port or intake slot of the respirator or ventilator, wherein after its insertion into the port or intake slot the cassette or a disc, which is operatively joined or connected to respirator or ventilator air intake tubes or vents, the device commences a controlled delivery of the anti-viral powders to the ventilator air delivery tubes, and therefore delivered by positive pressure air flow into the lungs of the patient. The exemplary respirator or ventilator is programmed to deliver the powders in the cassette over about a five to ten minute period. The first, used cassette or disc is removed, and a new disc or cassette in inserted about every two hours for next 6 to 48 hours, depending on the breathing capability and measured blood oxygen levels. After about 48 to 72 hour the patient's condition begins to improve.

### Example 4

A 52 year old male is using as a daily night-time sleeping routine an exemplary CPAP (continuous positive air pressure) device as provided herein for managing his sleep apnea. After he believes he has been exposed to an individual having COVID-19, before fitting the CPAP device's face mask for the night, he inserts a cassette or a disc containing anti-viral powders (as used or provided for in Example 2) in a receiving port or intake slot of the CPAP device, wherein after its insertion into the port or intake slot the cassette or a disc, which is operatively joined or connected to CPAP device air intake tubes or vents, the device commences a controlled delivery of the anti-viral powders to the ventilator air delivery tubes, and therefore delivered by positive pressure air flow into the lungs of the patient. The exemplary CPAP device is programmed to deliver the powders in the cassette over about a ten to twenty minute period.

### Example 5

A patient diagnosed with coronavirus, for example, having COVID-19, using products of manufacture, drugs or drug combinations and/or methods as provided herein is treated with:
(1) hydroxychloroquine (for example, PLAQUENIL™) is administered at a 400 bid (twice a day) loading dose on day one, the at 200 mg bid for the next nine or ten days; (2) azithromycin is administered (for example, ZITHROMAX™, or AZITHROCIN™) at a 500 mg bid loading dose on day one, then 500 mg in the morning (MANE) for days two, three and four, then azithromycin ceased and replaced by doxycycline (for example, DORYX™, DOXYHEXA™, DOXYLIN™) 100 mg bid for the remainder of the treatment (ten or eleven days), or
   azithromycin is first administered (for example, ZITHROMAX™, or AZITHROCIN™) at a 500 mg bid loading dose on day one, then 500 mg in the morning (MANE) for days two, three and four, then azithromycin ceased, and doxycycline 100 mg bid (for example, DORYX™, DOXYHEXA™, DOXYLIN™) every day for the full duration of the treatment (ten or eleven days, or more); and
(3) zinc sulfate, or zinc acetate, gluconate or picolinate, is administered at a dosage of 100 mg MANE every day of the treatment,
   wherein optionally the treatment lasts between about 10 days and 3 weeks, or 11 days and 2 weeks, or for about 10, 11, 12, 13 or 14 days.

### Example 6

A patient diagnosed with coronavirus, for example, having COVID-19, using products of manufacture, drugs or drug combinations and/or methods as provided herein is treated with:
(1) oseltamivir (for example, TAMIFLU™) is administered 75 mg three times a day (tid, or tds), or oseltamivir is dosaged tid for a daily total amount of between about 225 mg per day to about 450 mg per day;
(2) ritonavir is administered 50 mg bid and lopinavir 200 mg bid, or lopinavir and ritonavir administered bid as one tablet, for example, in the form of a KALETRA™ tablet, or in the form of heat stable granules, optionally in the form of heat stable pediatric granules (dosage can be adjusted by using a heat stable pediatric granulated form of KALETRA™);
(3) bismuth subcitrate is administered 300 mg bid; and
(4) zinc sulfate is administered 100 mg MANE every day of the treatment,
   wherein optionally the treatment lasts between about 5 days and 3 weeks, or 6 days and 2 weeks, or for about 7, 8, 9, 10, 11, 12, 13 or 14 days.

### Example 7

A patient diagnosed with coronavirus, for example, having COVID-19, using products of manufacture, drugs or drug combinations and/or methods as provided herein is treated with:
(1) hydroxychloroquine (for example, PLAQUENIL™) is first administered at a 400 bid (twice a day) loading dose on day one, then is administered at 200 mg bid for the remainder of the treatment, wherein optionally the treatment lasts between about 5 days and 3 weeks, or 6 days and 2 weeks, or for about 7, 8, 9, 10, 11, 12, 13 to 14 days,
(2) doxycycline (for example, DORYX™, DOXYHEXA™, DOXYLIN™) is administered at 100 mg bid;
(3) ribavirin or tribavirin (or COPEGUS™, REBETOL™, or VIRAZOLE™) administered at 400 mg in the morning (MANE), and 600 mg at night (NOCTE);
(4) favipiravir (or T-705, avigan, or favilavir) 800 mg bid; and
(5) zinc sulfate 100 mg MANE,
   wherein optionally the treatment lasts between about 5 days and 3 weeks, or 6 days and 2 weeks, or for about 7, 8, 9, 10, 11, 12, 13 to 14 days.

### Example 8

In alternative embodiments, products of manufacture as provided herein comprise formulations comprising all or any of (for example, for co-formulation or co-administration of): hydroxychloroquine (optionally, PLAQUENIL™), ivermectin (optionally STROMECTOL™), moxidectin (optionally CYDECTIN™, EQUEST™, QUEST™), doxycycline (optionally, DORYX™, DOXYHEXA™, DOXYLIN™), zinc (Zn), vitamin (Vit) D3 and vitamin C, or any combination thereof, for example hydroxychloroquine, Vitamin C, Vitamin D (optionally cholecalciferol or vitamin D3), and Zinc. In alternative embodiments, all or any of these drugs can be administered in the same products of manufacture or device, or using different products of manufacture or devices, and all or any of these drugs also can be, or alternatively can be, administered orally or by another means of administration, for example, IV, IM or sublingually.

In alternative embodiments, products of manufacture as provided herein comprise formulations comprising all or any of (for example, for co-formulation or co-administration of): hydroxychloroquine (optionally, PLAQUENIL™), azithromycin (optionally, ZITHROMAX™, or AZITHROCIN™, optionally an oral extended- or delayed-release formulation of azithromycin, or ZMAX™)), vitamin C, vitamin D (optionally cholecalciferol or vitamin D3), and zinc, or any combination thereof. In alternative embodiments, all or any of these drugs can be administered in the same products of manufacture or device, or using different products of manufacture or devices, and all or any of these drugs also can be, or alternatively can be, administered orally or by another means of administration, for example, IV, IM or sublingually.

In alternative embodiments, any or all of this therapeutic combination is administered orally and/or by inhalation (for example, by use of a nebulizer or equivalent), for example, ivermectin can be inhaled and the remainder of the drug combination is taken orally.

In alternative embodiments, provided products of manufacture comprising all or any of the exemplary formulations as set forth below, which can be administered as described in Arm A and Arm B, which are separate exemplary treatment regimens, where the numbers are in milligrams (mgs), and each column represents a day (i.e., the first column is day 1, the last column is day 10):

TABLE 1
Arm A dosage regimen.

| | Dosage (mg) | | | | | | | | | |
|---|---|---|---|---|---|---|---|---|---|---|
| Arm A | Day 1 | Day 2 | Day 3 | Day 4 | Day 5 | Day 6 | Day 7 | Day 8 | Day 9 | Day 10 |
| Hydroxychloroquine | 800 | 400 | 400 | 400 | 400 | 400 | 400 | 400 | 400 | 400 |
| Ivermectin | 12 | | | | | | 12 | | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |

TABLE 2
Arm B dosage regimen.

| | Dosage (mg) | | | | | | | | | |
|---|---|---|---|---|---|---|---|---|---|---|
| Arm B | Day 1 | Day 2 | Day 3 | Day 4 | Day 5 | Day 6 | Day 7 | Day 8 | Day 9 | Day 10 |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |
| Ivermectin | 12 | | | | | | 12 | | | |
| Bismuth | 1048 | 1048 | 1048 | 1048 | 1048 | 1048 | 1048 | 1048 | 1048 | 1048 |
| subsalicylate | | | | | | | | | | |

### Example 9: Exemplary Treatment Regimens

A 42 year (y) old female patient is first treated with osteltamivir (or TAMIFLU™) from the time she was in contact with a patient from China later found to be positive for a coronavirus, in particular, the COVID-19 virus (or so-called Wuhan coronavirus). She is treated for 4 days but then develops fever, cough, aches and some dyspnea, with blood result coming back positive for COVID-19 virus.

Because the patient remains positive for 2019-nCoV virus, treatment with lopinavir and ritonavir, or KALETRA™, ALTERA™, ALUVIA™, KALMELTREX, LOPIMUNE™ or LOPINAVIR™, is now added to the osteltamivir treatment, and within 48 hours her blood test becomes negative for the coronavirus.

This patient was quite ill reaching the dyspnoea phase and difficulty walking, and but for this new drug combination treatment would have been expected to die. The combination of drugs as provided herein can rapidly clear the coronavirus from the patient's blood, thereby terminating or significantly ameliorating an otherwise life-threatening illness.

### Example 10: Exemplary Treatment Regimen

A 47-year-old male returning from a trip develops draggles and muscle pains and a temperature of 37.5 C. He is tested for coronavirus and is found positive for COVID-19 on a swab test. He is commenced on a combination of twice-daily chloroquine to 250 mg, lopinavir 200 mg bid, retinovir 50 mg bid. together with 75 mg bid oseltamivir (optionally, TAMIFLU™).

His condition is further resolved within three days and he is negative on the swab test on day six. Muscle pains also disappear and he feels well with and is able to be released to go home from hospital on day seven.

### Example 11: Exemplary Treatment Regimen

A group of elderly travelers board a ship on a cruise, whereupon numerous individuals on the ship contract coronavirus COVID-19 on testing. This group is administered as a prophylactic therapy/treatment inhalant agents comprising two inhaled or aerosol doses, or twice daily, of 125 mg of chloroquine. None contract COVID-19 coronavirus and test negative for the virus upon arrival home after the cruise.

### Example 12: Exemplary Treatment Regimen

A 65-year-old female patient develops a respiratory infection with shortness of breath, and is admitted to a hospital, and is tested positive for COVID-19 coronavirus. She is treated with intravenous (IV) remdesivir 10 mg per kilogram, chloroquine 250 mg twice daily, inhaled interferon, and Kaletra™ (a lopinavir/ritonavir combination) 50/200 mg.

Over the next week the patient's shortness of breath first worsens, but then improves, having fewer muscle aches and shortness of breath. By day 8 her viral detection is negative for COVID-19. The patient is discharged on day 12 fully cured of the coronavirus using this exemplary drug combination.

### Example 13: Exemplary Treatment Regimen

Two Italian patients (one female, one male 69 years old with previous pulmonary disease) traveling in India tested positive for coronavirus COVID-19; they were administered lopinavir 200 mg twice daily, ritonavir 50 mg twice daily, chloroquine 250 mg twice daily, 75 mg oseltamivir (TAMIFLU™) twice daily, and the female patient tested negative after 7 days of this combined drug therapy, and the male patient showed significant improvement, with COVID-19 viral load diminished. No significant side effects from the administered drug combination were seen.

### Example 14: Exemplary Treatment Regimen

A 32-year-old male patient acquires nose swab positive coronavirus COVID-19 infection, possibly at a party. Symptoms include loss of the sense of taste, muscle aches, fever of 38.9 C, sore throat cough and difficulty with breathing. He is seen by a COVID-19 specialist, who commences patient on a combination of opaganib or YELIVA™ tid at 200 mg or 300 mg per dose; hydroxychloroquine 200 mg twice daily; lopinavir 200 mg three times per day; ritonavir 50 mg three times per day, and oseltamivir 75 mg tid, where one, several or all of these drugs are administered orally, IM, IV and/or by inhalation individually or in combination (for example, as a single formulation where applicable). The patient progressively loses his fever, regains the sense of taste, and after further five days the cough and sore throat improve.

Shortness of breath progressively improves but this requires more than 20 days (d) of continued treatment, yet the swabs are negative for COVID-19 on consecutive days by day 8.

### Example 15: Exemplary Treatment Regimen

A 72-year-old gentleman with a chronic cough suddenly develops fever, worsening of cough and shortness of breath walking up hills. He goes to the emergency room but the antibiotic that is given does not stop the progression of the illness. He sees a specialist who deals with respiratory disease and is found to be positive for COVID-19. He is commenced on opaganib or YELIVA™ tid at 200 mg or 300 mg per dose; and hydroxychloroquine 200 mg tid. He continues on this therapy and notices improvement within two days; a swab is negative on day seven and he is completely asymptomatic by day 20.

### Example 16: Exemplary Treatment Regimen

A 27-year-old female patient has a nose swab positive coronavirus COVID-19 infection. Symptoms include myalgia, sore throat cough and difficulty with breathing. She also complains of marked fatigue. The doctor commences her on a combination of hydroxychloroquine 200 mg twice daily (bid), lopinavir 200 mg three times per day (tid), ritonavir 50 mg tid, and oseltamivir 75 mg tid. The patient loses her fever after four days although the cough and sore throat continues for another two days. Shortness of breath progressively improves and after 12 days of treatment the swabs become negative on consecutive days.

### Example 17: Exemplary Treatment Regimen

A 73-year-old male develops loss of sense of smell and taste, followed by muscle aches and pains fever of 38.5° C. cough and a sore throat. He then notices he was short of breath and saw his doctor. The doctor commences him on hydroxychloroquine 200 mg twice daily increasing after three days to 3 times daily, together with azithromycin 50 mg three times a day, and within three days the patient's fever improves, then disappears. The patient continues on this composition of the two drugs for a total of 12 days and subsequent swabs are negative for coronavirus COVID-19.

### Example 18: Exemplary Treatment Regimen

A 49-year-old patient with nasopharyngeal swab proving an infection with coronavirus COVID-19, is treated to reduce symptoms using a combination of opaganib or YELIVA™ tid at 200 mg or 300 mg per dose, hydroxychloroquine 200 mg tid, azithromycin 50 mg bid. On day 4 of the treatment nasal swabs show an absence of the coronavirus, and this is also shown daily until day 14 showing a cure is achieved. The patient's symptoms abate fairly rapidly and he eventually becomes completely normal and asymptomatic.

### Example 19: Exemplary Prophylactic Treatment Regimens

Individuals are given an initial loading dosage of chloroquine, chloroquine phosphate, chloroquine diphosphate and/or hydroxychloroquine, which is administered or started at a high dose (for example, the so-called “loading dose”) for example, an oral (for example, a single dose such as a single tablet, capsule or pill), IV or IM dosage of between about 250 mg, 300 mg, 350 mg, 300 mg or 0.5 gram (gm)(500 mg) and 1.5 gm, or between about 400 mg and 1 gm, optionally with follow up administrations every 4 to 10 days, or every 1, 2, 3, 4, 5, 6, 7, 8, 9, 10 days or up to 20 or more days, at a lower dosage of between about 50 gm to 200 mg, or about 100 mg, total daily dosage, optionally continuing for between about one week to one month, and the chloroquine, chloroquine phosphate, chloroquine diphosphate and/or hydroxychloroquine is administered together with:
a macrolide drug, optionally azithromycin, and optionally the macrolide drug is started with a high dose (for example, a so-called “loading dose”), optionally an oral (for example, a single dose such as a single tablet, capsule or pill), IV or IM dosage of between about 400 mg to 0.5 gram (gm)(500 mg) and 1 gm, or at about 500 mg, optionally with follow up administrations every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month, and/or
opaganib or YELIVA™, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage,
and optionally the opaganib, or YELIVA™ is also administered or formulated with an antibiotic (optionally azithromycin or doxycycline), ivermectin (optionally at 12 mg ivermectin, optionally administered on days 1, 3, 6 and 8), hydroxychloroquine (optionally, PLAQUENIL™) and/or zinc (optionally zinc sulfate, optionally at (50 mg daily), and/or
lopinavir combined (formulated) with ritonavir, or KALETRA™, ALTERA™, ALUVIA™, KALMELTREX, LOPIMUNE™ or LOPINAVIR™.

### Example 20

A patient diagnosed with coronavirus, for example, having COVID-19, using products of manufacture, drugs or drug combinations and/or methods as provided herein is treated with:
(1) hydroxychloroquine (optionally, PLAQUENIL™) is administered at a 400 bid (twice a day) loading dose on day one, the at 200 mg bid for the next nine or ten days; (2) azithromycin is administered (optionally, ZITHROMAX™, or AZITHROCIN™) at a 500 mg bid loading dose on day one, then 500 mg in the morning (MANE) for days two, three and four, then azithromycin ceased and replaced by doxycycline (optionally, DORYX™, DOXYHEXA™, DOXYLIN™) 100 mg bid for the remainder of the treatment (ten or eleven days), or
   azithromycin is first administered (optionally, ZITHROMAX™, or AZITHROCIN™) at a 500 mg bid loading dose on day one, then 500 mg in the morning (MANE) for days two, three and four, then azithromycin ceased, and doxycycline 100 mg bid (or between about 25 to 500 mg bid) (optionally, DORYX™, DOXYHEXA™, DOXYLIN™) every day for the full duration of the treatment (ten or eleven days, or more); and
(3) zinc sulfate is administered at a dosage of 100 mg MANE every day of the treatment,
wherein optionally the treatment lasts between about 10 days and 3 weeks, or 11 days and 2 weeks, or for about 10, 11, 12, 13 or 14 days.

### Example 21

A patient diagnosed with coronavirus, for example, having COVID-19, using products of manufacture, drugs or drug combinations and/or methods as provided herein is treated with:
(1) oseltamivir (optionally, TAMIFLU™) is administered 75 mg three times a day (tid, or tds), or oseltamivir is dosaged tid for a daily total amount of between about 225 mg per day to about 450 mg per day;
(2) ritonavir is administered 50 mg bid and lopinavir 200 mg bid, or lopinavir and ritonavir administered bid as one tablet, optionally, in the form of a KALETRA™ tablet, or in the form of heat stable granules, optionally in the form of heat stable pediatric granules (dosage can be adjusted by using a heat stable pediatric granulated form of KALETRA™);
(3) bismuth subcitrate is administered 300 mg bid; and
(4) zinc sulfate is administered 100 mg MANE every day of the treatment,
wherein optionally the treatment lasts between about 5 days and 3 weeks, or 6 days and 2 weeks, or for about 7, 8, 9, 10, 11, 12, 13 or 14 days.

### Example 22

A patient diagnosed with coronavirus, for example, having COVID-19, using products of manufacture, drugs or drug combinations and/or methods as provided herein is treated with:
(1) hydroxychloroquine (optionally, PLAQUENIL™) is first administered at a 400 bid (twice a day) loading dose on day one, then is administered at 200 mg bid for the remainder of the treatment, wherein optionally the treatment lasts between about 5 days and 3 weeks, or 6 days and 2 weeks, or for about 7, 8, 9, 10, 11, 12, 13 to 14 days,
(2) doxycycline (optionally, DORYX™, DOXYHEXA™, DOXYLN™) is administered at between about 25 to about 600 mg bid, or between about 50 to about 500 mg bid, between about 100 to about 500 mg bid, or about 100 mg bid;
(3) ribavirin or tribavirin (or COPEGUS™, REBETOL™, or VIRAZOLE™) administered at 400 mg in the morning (MANE), and 600 mg at night (NOCTE);
(4) favipiravir (or T-705, avigan, or favilavir) 800 mg bid; and
(5) zinc sulfate 100 mg MANE,
wherein optionally the treatment lasts between about 5 days and 3 weeks, or 6 days and 2 weeks, or for about 7, 8, 9, 10, 11, 12, 13 to 14 days.

### Example 23

In alternative embodiments, provided are formulations or methods of administration of drug regimens comprising co-formulation or co-administration of: hydroxychloroquine (optionally, PLAQUENIL™), an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; zinc (Zn); vitamin (Vit) D3; and, vitamin C, or any combination thereof, for example hydroxychloroquine, Vitamin C, Vitamin D (optionally cholecalciferol, vitamin D3 or calcifediol), and Zinc.

In alternative embodiments, provided are formulations or methods of administration of drug regimens comprising co-formulation or co-administration of hydroxychloroquine (optionally, PLAQUENIL™), azithromycin (optionally, ZITHROMAX™, or AZITHROCIN™, optionally an oral extended- or delayed-release formulation of azithromycin, or ZMAX™)), vitamin C, vitamin D (optionally cholecalciferol, vitamin D3 or calcifediol), and zinc, or any combination thereof.

In alternative embodiments, any or all of this therapeutic combination is administered orally and/or by inhalation (for example, by use of a nebulizer or equivalent), for example, ivermectin can be inhaled and the remainder of the drug combination is taken orally.

In alternative embodiments, provided are exemplary formulations or methods of administration of drug regimens as set forth below (Arm C and Arm D being separate exemplary treatment regimens), where the numbers are in milligrams (mgs), and each column represents a day (i.e., the first column is day 1, the last column is day 10):

| ARM C (option 1) | day 1 | day 2 | day 3 | day 4 | day 5 | day 6 | day 7 | day 8 | day 9 | day 10 |
|---|---|---|---|---|---|---|---|---|---|---|
| Hydroxycloroquine | 800 | 400 | 400 | 400 | 400 | 400 | 400 | 400 | 400 | 400 |
| Ivermectin | 12 | | | | | | 12 | | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |

or, and alternative ARM C has 12 mg ivermectin administered on days 1, 3, 6 and 8:

| ARM C (option 2) | day 1 | day 2 | day 3 | day 4 | day 5 | day 6 | day 7 | day 8 | day 9 | day 10 |
|---|---|---|---|---|---|---|---|---|---|---|
| Hydroxycloroquine | 800 | 400 | 400 | 400 | 400 | 400 | 400 | 400 | 400 | 400 |
| Ivermectin | 12 | | 12 | | | 12 | | 12 | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |

| ARM D | day 1 | day 2 | day 3 | day 4 | day 5 | day 6 | day 7 | day 8 | day 9 | day 10 |
|---|---|---|---|---|---|---|---|---|---|---|
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |
| Ivermectin | 12 | | | | | | 12 | | | |
| Bismuth | 1048 | 1048 | 1048 | 1048 | 1048 | 1048 | 1048 | 1048 | 1048 | 1048 |
| subsalicylate | | | | | | | | | | |

### Example 24

A 67-year-old gentleman with cough fevers muscle pain and oximetry of 95% (which fell from 97%) presents for treatment because of a swab-positive Covid 19 test. He is given colchicine (or COLCRYS™, or MITIGARE™) 0.5 mg dose daily for 14 days together with hydroxychloroquine (or PLAQUENIL™)) 200 mg twice daily. He is also given zinc, for example, zinc picolinate, 50 mg each day for the 14 days. No antibiotic is used. 2 days after commencing the treatment his oximetry shows him to be 9600 and by day four return to 97%.

The next 7 days his cough improves, fevers disappeared, but muscle aches and pains take somewhat longer to disappear.

### Example 25

In another example, the wife of an intensivist who was previously positive for Covid 19 starts developing symptoms of the infection herself, which may well have been acquired at home. Cough is her dominant symptom and she also loses her ability to taste and smell. Her oximeter reading remains at 96%, there having been no previous reading. She commences colchicine (or COLCRYS™, or MITIGARE™) 0.5 mg twice daily for 14 days together with ivermectin 6 mg twice daily for the 1^{st} 5 days and thereafter 6 mg each morning. Her cough takes 2 weeks to reverse but she feels much better and more energetic within 5 days of treatment and her sense of smell and taste starts to return around the same time.

### Example 26

In another example, a 71 year male old patient had been taking colchicine 0.6 mg twice daily for a long-standing constipation; this treatment is a well proven and trialed therapy. He was also diabetic, hypertensive, and had mild congestive heart failure. He developed a flulike illness with cough, sore throat, muscle aches and pains, with later added fevers and ultimately he lost his sense of smell after about 4 or 5 days. Standard treatment for his cold did not improve the symptoms, and he underwent a swab test to look for Covid 19. This test turned out to be positive and the patient had added to his colchicine treatment comprising hydroxychloroquine 200 mg twice daily together with ivermectin 12 mg on day 1, 3, 6 and 8. At this stage his oximeter reading fell to 92% and he was quite dyspneic. In 2 days of commencing therapy noticed that his fever had gone but his cough continued. At this time zinc sulphate was added with 220 mg/day with azithromycin 250 mg each morning for 5 days. The patient experienced marked improvement with his oximeter reading rising to 95% and later to 97%. His ‘cold’ symptoms kept on improving with the cough resolved by day 14. His subsequent throat swab was negative.

### Example 27

In another example, a colchicine (or COLCRYS™, or MITIGARE™) as used in the above two examples is co-formulated or co-administered with hydroxychloroquine (optionally, PLAQUENIL™) and/or an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; and optionally colchicine (or COLCRYS™, or MITIGARE™) also is co-formulated or co-administered with zinc (Zn); vitamin (Vit) D3; and, vitamin C, or any combination thereof, for example colchicine (or COLCRYS™, or MITIGARE™) with or without hydroxychloroquine, Vitamin C, Vitamin D (optionally cholecalciferol, vitamin D3 or calcifediol) and/or Zinc.

### Example 28

An elderly patient aged 74 was admitted to hospital because of shortness of breath. The oximeter reading was 91%. His daughter contacted the clinic to try and ask doctors in the hospital to treat the patient using hydroxychloroquine-based therapy. Because it took a long time to convince the prescribing physician in the hospital to start such treatment, the daughter was given the medication which she obtained from the local pharmacy. The patient was already being given intravenous remdesivir but had not responded to this. His new treatment was the continuation of remdesivir but hydroxychloroquine 200 mg twice daily (bid, or BD) was added to the treatment. He was also given azithromycin 250 mg each morning for the 1^{st} 5 days and zinc sulphate 50 mg (base) daily for the entire 10 day treatment. His improvement was rapid and within the 1^{st} 2 days his oximeter reading rose to 95%. The intravenous treatment was ceased he continued for the entire 10 days of hydroxychloroquine in sync with the 1^{st} 5 days with azithromycin. His ECG monitoring showed no abnormalities. He was discharged home with an oximeter reading of 96%.

### Example 29

In another example, a performer aged 56 became infected at home from her infected family. Her illness was characterized by cold-like symptoms with cough, sneezing, fever, muscle aches, pains and the loss of the sense of smell. Her oximeter reading did not fall below 96% but she was nevertheless quite dyspnoeic at rest. Being allergic to azithromycin she was treated as the elderly patient aged 74, discussed above; however, hydroxychloroquine 200 mg twice daily (bid, or BD) but instead of azithromycin she was given doxycycline 100 mg twice daily gather with the zinc sulphate (50 mg daily). The entire treatment was continued for 10 days and by day 8 most of her Covid 19 symptoms disappeared. Her original throat swab was positive but later on by day 14 her Covid 19 swab was now negative.

### Example 30

In another example, two patients with positive throat swabs for Covid 19 were treated with hydroxy chloroquine 200 mg twice daily and instead of azithromycin or doxycycline they were given clarithromycin 250 mg twice daily. The clarithromycin was continued for the full 10 days of treatment together with zinc sulphate 50 mg each morning. There were no ECG abnormalities. In addition to these medications the patient also had daily 5000 units of vitamin D3 as well as daily 1000 units of vitamin C throughout the therapy. They were both throat swab negative by day 12.

### Example 31: Ivermectin-Containing Combinations

78 patients were treated with the singular 10 day protocol consisting of ivermectin 12 mg taken on day 1, 3 and 8. They were also treated with doxycycline 100 mg twice daily for the entire 10 day period. They were also treated with zinc sulphate 220 mg each morning combined with vitamin D3 5000 units and vitamin C 1000 units each taken in the morning. They were all swab Covid 19 positive for between 2 and 7 days before they were commenced on treatment.

Various Covid 19 symptoms were present in this cohort, and lowered oximeter readings were seen down to 86%, which progressively reverted to normality at various times during the treatment. All patients were cured of Covid 19 as determined by the swab test. Even those who were quite ill, breathless, severely fatigued, and had low oximeter readings. Some had external treatment using hydroxychloroquine alone. The addition of the ivermectin protocol as described above turned these patients around very quickly towards normality. 34 of these patients were wearing monitoring devices which were sending ECG traces back to base and none developed any abnormal arrhythmias while on the triple anti-Covid 19 protocol.

### Example 32

In another example, 288 patients were treated using a combination of hydroxychloroquine 200 mg twice a day for 10 days, with the 1^{st} 5 days on azithromycin 250 mg twice daily. In addition to these drugs they were also on treatment using daily both 5000 IU vitamin D3 and vitamin C 1000 mg. Each of these was taken daily for 10 days. The patients were naso-pharyngeal swab positive for Covid 19. They commenced treatment from between 2 and 9 days after initial diagnosis by swab. All had the classic symptoms of Covid 19 infection in various degrees of severity but none was admitted to hospital. The symptoms included fever, cough, muscular pains, breathlessness, fatigue, and in a small proportion of these patients there was loss of taste and loss of smell symptoms. Some also had diarrhoea.

About 6 of these patients reversed their infection as judged by a negative swab at 14 days although some had swabs done at between 10 to 14 days. 6 were deemed to have failed to be cured because they continued to be symptomatic and had a positive swab test. They were then treated with the ivermectin triple therapy (ivermectin 12 mg taken on day 1, 3 and 8; doxycycline 100 mg twice daily for a 10 day period; zinc sulphate 220 mg each morning combined with vitamin D3 5000 units and vitamin C 1000 units each taken in the morning) and recovered.

### Example 33: Amantadine-Containing Combinations

A 65-year-old patient sought help because of his chronic cough over seven days, muscle aches and pains tiredness fevers and loss of sense of smell. He was commenced on amantadine 100 mg per day in the first two days and this was elevated to 100 mg twice daily for the next 10 days. He was also given colchicine 0.6 mg twice daily. On top of that he was also treated with oxygen therapy and vitamins D 5000 IU and Vitamin C 1 g/day. Under this treatment he improved in only three days and by day seven of the treatment in the longer had cough and muscle aches but was still better still very tired. He lost his cough at around day 14 and was able to return to work after three weeks.

### Example 34: Cyclosporin-Containing Combinations

A 47-year-old female nurse who worked in intensive care developed symptoms of COVID-19 infection. She developed a skin rash runny nose muscle pains cough and ultimately some breathlessness. Her oximeter reading was 92 and she was advised to be hospitalised. She chose to be treated by combination of cyclosporine at a dose of 3 mg/kg (180 mg daily) together with 12 mg ivermectin once plus zinc 50 mg base and doxycycline 100 mg bid, all for 10 days. She began the treatment when her oximeter reading was 92% and within three days the oximeter rose to 95% by day seven rose to 97%. She had minimal side-effects of loose motions and some cramping but her nasopharyngeal swab was negative on day 12.

A number of embodiments of the invention have been described. Nevertheless, it can be understood that various modifications may be made without departing from the spirit and scope of the invention. Accordingly, other embodiments are within the scope of the following claims.

1. A product of manufacture fabricated or manufactured as an inhaler, asthma “puffer-like” device, nebulizer or nasal spray device or equivalent, for delivering a medication or a drug,
and optionally the inhaler, asthma “puffer-like” device, nebulizer or nasal spray device or equivalent is a hand-held or otherwise portable device,
and optionally the device is a metered or dose-counting inhaler or a nasal spray device,
and optionally the inhaler, asthma “puffer-like” device, nebulizer or nasal spray device or equivalent further comprises a cassette, packette, interchangeable disk (for example, for holding a powder) or reservoir (optionally a refillable reservoir) in or on the product of manufacture, or a removable cassette or packette or, interchangeable disk (for example, for holding a powder) that can be inserted into a slot or port on the product of manufacture, or a separate reservoir or container operatively connected, linked or joined to the product of manufacture, that comprises the drug or the medication,
and optionally the inhaler, asthma “puffer-like” device, nebulizer or nasal spray device or equivalent is designed, manufactured or configured to deliver a powder or a liquid aerosol. spray or mist,
and optionally the cassette, packette or interchangeable disk (for example, for holding a powder) comprises an adjustable value that modulates or regulates the amount of the medication or drug delivered to a user of the inhaler, asthma “puffer-like” device, nebulizer or nasal spray device or equivalent,
and optionally the product of manufacture comprises no filter, and optionally the product of manufacture comprises at least one air filter, optionally a high-efficiency particulate air (HEPA) filter or a high-efficiency particulate absorbing and high-efficiency particulate arrestance air filter, or an air filter capable of filtering out airborne viruses,
and optionally the product of manufacture further comprises, or is operatively connected to, an air ionizer, or a device capable of making or generating electrons and/or negatively-charged oxygen ions and/or positively-charged ions, and optionally this air ionizer or device capable of generating electrons and/or ions is configured in the product of manufacture or is operatively connected to the product of manufacture such that the ionized air, or air comprising the electrons and/or ions, is delivered to a patient, optionally as a forced air or positive air pressure flow of air, to or into the nose and or mouth, and optionally to the trachea, bronchi or lungs, of a user.

2. A product of manufacture fabricated or manufactured as medical device capable of delivering:
(a) hot or warm or humidified air, optionally as a forced air or positive air pressure flow of air, to or into the nose and or mouth, and optionally to the trachea, bronchi or lungs, of a user, and/or
(b) ionized air, or air comprising the electrons and/or negatively-charged oxygen ions and/or positively-charged ions, to or into the nose and or mouth, and optionally to the trachea, bronchi or lungs, of a user,
wherein optionally the medical device is fabricated or manufactured as a hand-held device, and optionally the medical device is fabricated or manufactured as a respirator or ventilator, a CPAP (continuous positive air pressure) device (optionally for managing or treating sleep apnea), an inhaler, an asthma puffer or a nebulizer,
and optionally the product of manufacture delivers the forced air or positive air pressure flow of air, or warm or hot and/or humidified air, or room temperature air, and/or ionized air, directly into the user's mouth and/or nose, and optionally the forced air or positive air pressure flow of air, and/or the room temperature, hot or warm air, is delivered or permeates through the nose into the sinuses and turbinates, down the trachea, and into the main bronchi and bronchioles,
and optionally the product of manufacture comprises controls or dials to set or adjust the warmth or heat of the air and/or to adjust the force the air is delivered into the nose or mouth, and bronchi and lungs of the user,
and optionally the product of manufacture is battery powered,
and optionally the product of manufacture comprises a mouthpiece or facemask (optionally a disposable mouthpiece or facemask) to fit or place the device over the mouth and/or nose for forced air delivery, and optionally the mouthpiece or facemask can fit over both the nose and the mouth for simultaneous air delivery to or into both nose and mouth,
and optionally the product of manufacture comprises a refillable, renewable and/or disposable cassette, packette, interchangeable disk (for example, for holding a powder) or drug-comprising module disposed onto or in the medical device, or a removable cassette or packette, interchangeable disk (for example, for holding a powder) that can be inserted into a slot or port on the product of manufacture, or a separate reservoir or container operatively connected, linked or joined to the product of manufacture, and optionally the refillable, renewable and/or disposable cassette, packette, interchangeable disk (for example, for holding a powder) or drug-comprising module comprises a drug or the medication,
and optionally the drug or medication comprises an asthma or an anti-microbial (for example, antiviral) drug, or an antibiotic agent or drug (optionally gentamycin), a biofilm dissolving agent (optionally PULMOZYME™), an anticholinergic (optionally tiotropium bromide or SPIRIVA™), a bronchodilator, steroid, a corticosteroid, a antileukotriene, or an asthma drug (optionally formoterol, salbutamol, albuterol, or VENTOLIN™), an antifungal agent, itraconazole (for example, SPORANOX™, SPORAZ™, ORUNGAL™), an anti-inflammatory agent or any formulation or drug combination of any of the preceding claims,
and optionally the product of manufacture comprises at least one air filter, optionally a high-efficiency particulate air (HEPA) filter or a high-efficiency particulate absorbing and high-efficiency particulate arrestance air filter, or an air filter capable of filtering out airborne viruses,
and optionally the product of manufacture further comprises or is operatively connected to an air ionizer, or a device capable of making or generating electrons and/or negatively-charged oxygen ions and/or positively-charged ions, and this air ionizer or device capable of generating electrons and/or ions is operatively connected to the product of manufacture such that the ionized air, or air comprising the electrons and/or ions, is delivered to a patient, optionally as a forced air or positive air pressure flow of air, to or into the nose and or mouth, and optionally to the trachea, bronchi or lungs, of a user, and optionally the air ionizer, or a device capable of making or generating electrons and/or negatively-charged oxygen ions and/or positively-charged ions, is connected to the inhalation port of a ventilator as provided herein.

3. The product of manufacture of any of the preceding claims, wherein the drug or medication comprises: a therapeutic combination of drugs or a drug, a pharmaceutical dosage form, a drug delivery device, or a product of manufacture, comprising:
(a) opaganib or YELIVA™; or opaganib or YELIVA™ and oral and/or inhaled or aerosol chloroquine (or ARALEN™), chloroquine phosphate, quinine, chloroquine diphosphate and/or hydroxychloroquine (for example, PLAQUENIL™); or opaganib, hydroxychloroquine and azithromycin (for example, ZITHROMAX™, or AZITHROCIN™, optionally an extended-release formulation of azithromycin, or ZMAX™),
wherein optionally each or both of the opaganib and the chloroquine (or ARALEN™), chloroquine phosphate, quinine, chloroquine diphosphate and/or hydroxychloroquine (for example, PLAQUENIL™) and/or azithromycin are in or formulated as a formulation for inhalation, for example, formulated as an aerosol, liquid or powder, or each or both are formulated for oral, intramuscular or intravenous administration,
wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage;
(b) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 300 mg per day,
optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder;
(c) lopinavir, ritonavir and oseltamivir (for example, TAMIFLU™), and/or zanamivir (RELENZA™);
(d) lopinavir combined (formulated) with ritonavir (optionally NORVIR™), or the combination lopinavir and ritonavir, or KALETRA™, ALTERA™, ALUVIA™, KALMELTREX, or LOPIMUNE™, and/or zanamivir (RELENZA™), and optionally the lopinavir, ritonavir and/or zanamivir are separately formulated;
(e) lopinavir combined (formulated) with ritonavir (or KALETRA™, ALTERA™, ALUVIA™, KALMELTREX or LOPIMUNE™), or lopinavir and ritonavir, and oseltamivir (for example, TAMIFLU™), and/or zanamivir (RELENZA™), optionally also with inhaled or aerosol formulations or versions of chloroquine (or ARALEN™), chloroquine phosphate, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) simultaneously;
(f) lopinavir, ritonavir (optionally NORVIR™), chloroquine and oseltamivir (or TAMIFLU™), and/or zanamivir (RELENZA™); wherein optionally the chloroquine comprises inhaled or aerosol chloroquine (or ARALEN™), chloroquine phosphate, quinine, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) simultaneously;
(g) lopinavir and oseltamivir (for example, TAMIFLU™), and/or zanamivir (RELENZA™)
(h) ritonavir (optionally NORVIR™) and oseltamivir (for example, TAMIFLU™), and/or zanamivir (RELENZA™);
(i) remdesivir (for example, GS-5734™, Gilead Sciences) alone, or oseltamivir (for example, TAMIFLU™) and remdesivir (for example, GS-5734™, Gilead Sciences), and optionally the remdesivir is an oral formulation and/or an inhaled or aerosol remdesivir formulation;
(j) oseltamivir (for example, TAMIFLU™) and efavirenz (for example, SUSTIVA™), and/or zanamivir (RELENZA™);
(k) oseltamivir (for example, TAMIFLU™) and nevirapine (or the combination efavirenz with emtricitabine and tenofovir (optionally tenofovir disoproxil or VIREAD™), or ATRIPLA™);
(l) oseltamivir (or TAMIFLU™) and amprenavir (for example, AGENERASE™);
(m) oseltamivir (for example, TAMIFLU™) and nelfinavir (for example, VIRACEPT™); or
(n) a thiazolide class drug, optionally nitazoxanide (or ALINIA™, NIZONIDE™) or tizoxanide (or 2-Hydroxy-N-(5-nitro-2-thiazolyl)benzamide), with or in combination with any of (a) to (i), or the thiazolide class drug (for example, nitazoxanide or tizoxanide) and oseltamivir (or TAMIFLU™);
(o) plitidepsin (also known as dehydrodidemnin B), or APLIDIN™ (PharmaMar, S.A.);
(p) an inhibitor or S-phase kinase-associated protein 2 (SKP2), or dioscin, or niclosamide, or NICLOCIDE™, FENASAL™, or PHENASAL™;
(q) ribavirin or tribavirin (or COPEGUS™, REBETOL™, or VIRAZOLE™), interferon beta 1b, or a combination of ribavirin and interferon beta, or a combination of lopinavir and ritonavir (optionally NORVIR™) and interferon-beta-1b;
(r) abacavir, acyclovir for example, (ACICLOVIR™), adefovir, amantadine, ampligen, amprenavir (for example, AGENERASE™), aprepitant, arbidol, atazanavir, tenofovir, a combination of efavirenz and emtricitabine and tenofovir (or ATRIPLA™), balavir, baloxavir marboxil (XOFLUZA™), bepotastine (or TALION™, BEPREVE™), bevirimat, bictegravir, biktarvy, brilacidin, cidofovir, caspofungin, lamivudine and zidovudine (for example, COMBVIR™), cobicstat, colisitin, cocaine, darunavir, delavirdine, descovy, didanosine, docosanol, dolutegravir, ecoliever, edoxudine, efavirenz (optionally, SUSTIVA™), elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, molnupiravir, moroxydine, nelfinavir (or VIRACEPT™), nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430, EIMIUCILLIN-A™), remdesivir (for example, GS-5734™, Gilead Sciences), cytarabine, gemcitabine, emtricitabine, lamivudine, zalcitabine, abacavir, acyclovir, entecavir, stavudine, telbivudine, idoxuridine and/or trifluridine or any combination thereof), oseltamivir (or TAMIFLU™), peginterferon alfa-2a, penciclovir, peramivir (for example, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir (optionally NORVIR™), saquinavir, sofosbuvir (optionally SOFORAL™, SOLVALDI™), stavudine, telaprevir, tegobuv, tenofovir (optionally tenofovir alafenamide or tenofovir disoproxil or VIREAD™), tipranavir, trifluridine, trizivir, tromantadine, truvada, valaciclovir (for example, VALTREX™), valganciclovir, valrubicin, vapreotide, vicriviroc, vidarabine, viramidine, velpatasvir, vivecon, zalcitabine, zanamivir (for example, RELENZA™), zidovudine, an immunosuppressive drug (optionally tocilizumab or atlizumab, or ACTEMRA™, or ROACTEMRA™) or any combination thereof;
(s) an mucolytic therapy or drug, optionally acetylcysteine, ambroxol, bromhexine, carbocisteine, erdosteine, mecysteine or dornase alfa, or an expectorant, optionally guaifenesin;
(t) a viral, or a coronavirus or a COVID-19, protease inhibitor, and optionally the protease inhibitor comprises: ASC09 (CAS registry no. 1000287-05-7) (Janssen Research and Development, LLC); lopinavir, ritonavir (or NORVIR™) or ASC09 and ritonavir; a JAK1/2 inhibitor (optionally baricitinib); compound 11r (University of Lubeck, Germany, see for example, Zhang et al J. Med Chem 2020, Feb. 11, 2020), PF-07321332 (PFIZER); PF-07304814 (PFIZER); or darunavir, cobicistat, darunaviror cobicistat;
(u) an angiotensin-converting enzyme 2 (ACE2) inhibitor, optionally to block the site of viral spike protein interaction for anti-SARS-CoV-2 infection control;
(v) an anti-vascular endothelial growth factor (VEGF) (optionally VEGF-A) drug or antibody, optionally bevacizumab;
(w) a protease inhibitor, optionally danoprevir, optionally a serine protease inhibitor, optionally or narlaprevir (optionally ARLANSA™), camostat or camostat mesilate (or FOIPAN™);
(x) anti-PD-1 checkpoint inhibitor, optionally camrelizumab;
(y) a compound or antibody capable of binding complement factor C5 and blocking membrane attack complex formation, optionally eculizumab;
(z) a cathepsin inhibitor, optionally a cathepsin K, B or L inhibitor, optionally relacatib;
(aa) thalidomide, or thalidomide and glucocorticoid (optionally low-dose glucocorticoid), or and thalidomide and celecoxib;
(bb) an antibacterial antibiotic, a macrocyclic lactone antibiotic, or a macrolide drug,
wherein optionally the macrolide drug comprises azithromycin (for example, ZITHROMAX™, or AZITHROCIN™, optionally an extended-release formulation of azithromycin, or ZMAX™), clarithromycin (for example, BIAXIN™), erythromycin (for example, ERYTHROCIN™), or fidaxomicin (for example, DIFICID™ or DIFICLIR™), troleandomycin (for example, TEKMISIN™), tylosin (for example, TYLOCINE™ or TYLAN™), solithromycin (for example, SOLITHERA™), oleandomycin (or SIGMAMYCINE™), midecamycin, roxithromycin, kitasamycin or turimycin, josamycin, carbomycin or magnamycin, and/or spiramycin, and optionally the macrocyclic lactone antibiotic comprises an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,
and optionally the antibacterial, optionally azithromycin (optionally, ZITHROMAX™, or AZITHROCIN™, optionally an oral extended-release formulation of azithromycin, or ZMAX™) or a tetracycline class drug such as doxycycline, is formulated and/or administered with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and cholecalciferol (vitamin D3),
and optionally the antibacterial antibiotic comprises an antimycobacterial drug, and optionally the antimycobacterial drug comprises clofazimine (optionally LAMPRENE™), optionally dosaged at about 100 mg per day, or between about 50 mg and 150 mg per day, and optionally also including colchicine;
and optionally the macrolide drug, optionally azithromycin, is administered at a dosage of about 100 to 200 mg a day, or at about 25 to 100 mg three times a day (tid), or at 50 mg tid,
wherein optionally the antibacterial antibiotic or the macrolide drug. optionally azithromycin, is formulated for normal release or as a delayed release formulation,
and optionally the antibacterial antibiotic comprises a tetracycline class drug comprising one or more of: a glycylcycline or a fluorocycline class drug, or an analogue thereof, and optionally the tetracycline class drug comprises: tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof;
(cc) chloroquine (or ARALEN™), chloroquine phosphate, quinine, chloroquine diphosphate and/or hydroxychloroquine (for example, PLAQUENIL™) with (or formulated with) or in combination with any of (a) to (cc), or chloroquine, chloroquine phosphate, quinine, chloroquine diphosphate and/or hydroxychloroquine (for example, PLAQUENIL™) and oseltamivir (or TAMIFLU™);
(dd) chloroquine (for example, ARALEN™), chloroquine phosphate, quinine, chloroquine diphosphate and/or hydroxychloroquine (for example, PLAQUENIL™) alone or with:
(i) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 10 mg to 80 mg or about 6 mg to 60 mg dosages, or 12 to 60 mg dosages, and/or
(ii) vitamin D, vitamin D2 (or ergocalciferol), vitamin D3 (or cholecalciferol) optionally at a dosage of between about 3,000 to 100,000 units per day, or
(iii) with (i) and (ii) and zinc (optionally a zinc sulphate, acetate, gluconate or picolinate) optionally at a dosage of between about 1 mg to 250 mg, or
(iv) the combination of (iii) also with a tetracycline class drug, wherein
optionally the tetracycline class drug comprises doxycycline, or DORYX™, DOXYHEXA™, DOXYLIN™, optionally dosages at between about 100 mg to 600 mg per day, optionally between about 200 mg to 400 mg;
(ee) colchicine, or COLCRYS™, MITIGARE™;
(ff) a corticosteroid class drug such as budesonide (optionally RHINOCORT™ or PULMICORT™), prednisolone (or ORAPRED™), methyl-prednisolone, prednisone (or DELTASONE™ or ORASONE™) or hydrocortisone (or CORTEF™),
and optionally the corticosteroid class drug (for example budesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,
and optionally the corticosteroid class drug is formulated as a powder or for administration in an inhaler or by nasal spray, or for rectal administration,
and optionally the corticosteroid class drug (for example, budesonide) is administered together with or in combination with 10 mg to 80 mg, an antibiotic (optionally azithromycin or a tetracycline class drug
and optionally the corticosteroid class drug (for example budesonide) is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, an antibiotic (optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™) or a tetracycline class drug, optionally doxycycline), zinc and/or a vitamin (optionally vitamin D, D2 (or ergocalciferol), D3 (or cholecalciferol or calcifediol), C, E, B12, B6);
(gg) an anti-androgen drug, optionally bicalutamide, optionally CASODEX™, and optionally the anti-androgen, optionally bicalutamide is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
(hh) a hydrocortisone or cortisol (optionally CORTEF™, SOLUCORTEF™), optionally hydrocortisone sodium succinate or hydrocortisone acetate, or dexamethasone (optionally DEXTENZA™, OZURDEX™, NEOFORDEX™);
(ii) an alpha-ketoamide (a-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2), and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin (optionally, ZITHROMAX™, or AZITHROCIN™, optionally an oral extended-release formulation of azithromycin, or ZMAX™) or a tetracycline class drug, optionally doxycycline (or DORYX™, DOXYHEXA™, DOXYLIN™)); chloroquine (or ARALEN™), chloroquine phosphate, chloroquine diphosphate and/or hydroxychloroquine (optionally, PLAQUENIL™); zinc; remdesivir (optionally, GS-5734™, Gilead Sciences); oseltamivir (or TAMIFLU™); and/or, hydrocortisone; or, any combination thereof;
(jj) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 240 mg per day,
optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated with and/or administered with chloroquine (or ARALEN™), chloroquine phosphate, chloroquine diphosphate and/or hydroxychloroquine (optionally, PLAQUENIL™) with or without zinc (optionally a zinc sulphate, acetate, gluconate or picolinate), and optionally this combination is administered weekly, or every two week, or one every 5 to 28 days, as a prophylactic,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered alone for 1, 2, 3, 4, 5, 6, 7, 8, 9 or 10 days or up to 20 more days, followed by administration of an antibiotic (optionally doxycycline) for a corresponding period of days, and optionally repeating the cycle of dosaging;
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated or administered with:
(i) at least one antibiotic (wherein optionally the antibiotic is doxycycline (optionally, DORYX™, DOXYHEXA™, DOXYLIN™) (optionally formulated or administered at a dosage of between about 25 mg to 500 mg), or azithromycin (optionally, ZITHROMAX™, or AZITHROCIN™, optionally an oral extended-release formulation of azithromycin, or ZMAX™) (optionally formulated or administered at a dosage of between an about 50 mg to 2000 mg);
(ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
(iii) a zinc (optionally a zinc sulphate, acetate, gluconate or picolinate) optionally formulated or administered at a dosage of between about 1 mg to 250 mg; and/or
(iv) at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin. is administered or formulated alone or in combination with any of the above (i) to (iv)(for example, at least one antibiotic, chloroquine (or ARALEN™), chloroquine phosphate, chloroquine diphosphate and/or hydroxychloroquine (optionally, PLAQUENIL™), zinc and/or at least one vitamin are formulated (and administered) as oral formulations (for example, as tablets, capsules, gels or geltabs), injectable formulations, powders (for example, for inhalation or for addition to an ingestible liquid) or liquids (for example, for ingestion, infusion or injection);
(kk) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin) or doramectin (optionally DECTOMAX™), and/or a tetracycline tetracycline class drug, and optionally the tetracycline class drug comprises doxycycline, or DORYX™, DOXYHEXA™, DOXYLIN™;
(ll) a dendrimer, optionally astodrimer sodium (Starpharma, Melbourne, Australia);
(mm) an antihistamine class drug such as azelastine, or ASTELIN™, OPTIVAR™, ALLERGODIL™, bepotastine (or TALION™, BEPREVE™), brompheniramine, fexofenadine or ALLEGRA™, pheniramine or AVIL™, or chlorpheniramine;
(nn) a selective serotonin reuptake inhibitor (SSRI) class drug, optionally fluvoxamine, or LUVOX™, FAVERIN™, FLUVOXIN™;
(oo) a peroxisome proliferator-activated receptor (PPAR) agonist, wherein optionally the PPAR agonist comprises fenofibrate, or TRICOR™, FENOBRAT™, FENOGLIDE™ or LIPOFEN™, optionally the PPAR agonist comprises a combination of fenofibrate and pravastatin, or PRAVAFENIX™,
(pp) any one or several or all of (a) to (nn) with (or formulated with or formulated as an) inhaled or aerosol (for example, powder or aerosol) formulation and/or formulated with or formulated as an oral, intramuscular (IM) or intravenous (IV) formulation, wherein optionally both the inhaled or aerosol and the oral, IV and/or IM formulations are administered simultaneously or sequentially,
and optionally the inhaled or aerosol formulation comprises chloroquine (or ARALEN™), chloroquine phosphate, quinine, chloroquine diphosphate and/or hydroxychloroquine (for example, PLAQUENIL™) and/or oral chloroquine (or ARALEN™), chloroquine phosphate, quinine, chloroquine diphosphate and/or hydroxychloroquine (for example, PLAQUENIL™) administered simultaneously or overlapping; and/or
(qq) any combination of (a) to (pp), optionally with or also formulated with at least one vitamin or mineral, wherein optionally the at least one vitamin or mineral comprises vitamin K, vitamin D (optionally Vitamin D3, optionally administered at about 1000 to 4000 ugm/day), vitamin B12, vitamin B6 (or pyridoxine), vitamin E, and/or vitamin C, which optionally are given or formulated for administration enterally or parenterally, and/or zinc (optionally a zinc sulphate, acetate, gluconate or picolinate, optionally administered at about 50 to 200 mg/day, or 100 mg/day), or a flavonoid, plant flavonol or quercetin optionally administered at between about 250 to 500 mg bid, or melatonin, and/or aviptadil (or INVICORP™); and/or
(rr) any combination of (a) to (qq),
and optionally any one of, any combination of or all of (a) to (rr) are formulated as an aerosol, a mist or a powder.

4. The product of manufacture of any of the preceding claims, wherein the drug or medication comprises or are formulated with) an (or an additional) anti-viral drug or medication, or anti-microbial drug, or palliative agent or drug,
wherein optionally the anti-viral drug or medication, or anti-microbial drug, is or comprises efavirenz (for example, SUSTIVA™), tenofovir (optionally tenofovir alafenamide or tenofovir disoproxil or VIREAD™), emtricitabine and tenofovir, nevirapine (or the combination efavirenz with emtricitabine and tenofovir, or ATRIPLA™), amprenavir (for example, AGENERASE™), nelfinavir (for example, VIRACEPT™) and/or remdesivir (for example, GS-5734™, Gilead Sciences), a viral RNA-dependent RNA polymerase inhibitor, optionally favipiravir (optionally AVIGAN™) or sofosbuvir (optionally SOVALDI™, SOFORAL™); or, an adenosine analog (optionally galidesivir, optionally IMMUCILLIN-A™),
and optionally the anti-viral drug or medication is or comprises an anti-retroviral drug or drug combination, and optionally the anti-retroviral drug or drug combination comprises: darunavir and cobicistat (for example, REZOLSTA™ or PREZCOBIX™); atazanavir and cobicistat (or EVOTAZ™); abacavir, lamivudine and dolutegravir (TRIUMEQ™); tenofovir (or disoproxil or emtricitabine or VIREAD™) and elvitegravir and cobicistat (for example, STRIBILD™); tenofovir (or disoproxil or emtricitabine) and elvitegravir and cobicistat (COMPLERA™ or EVIPLERA™); efavirenz (optionally, SUSTIVA™), emtricitabine and tenofovir (ATRIPLA); lamivudine, nevirapine and stavudine (for example, TRIOMUNE™); atazanavir and cobicistat (for example, EVOTAZ™); lamivudine and raltegravir (for example, DUTREBIS™); lamivudine and dolutegravir (or DOVATO™); doravirine, lamivudine and tenofovir (optionally tenofovir alafenamide or tenofovir disoproxil or VIREAD™) (for example, DELSTRIGO™); or lamivudine, zidovudine and nevirapine (for example, CUOVIR-N™), aviptadil (or INVICORP™), a macrolide and/or a macrocyclic lactone antibiotic (optionally the macrolide drug comprises azithromycin (for example, ZITHROMAX™, or AZITHROCIN™, optionally an oral extended-release formulation of azithromycin, or ZMAX™), clarithromycin (for example, BIAXIN™), erythromycin (for example, ERYTHROCIN™), or fidaxomicin (for example, DIFICID™ or DIFICLIR™), troleandomycin (for example, TEKMISIN™), tylosin (for example, TYLOCINE™ or TYLAN™), solithromycin (for example, SOLITHERA™), oleandomycin (or SIGMAMYCINE™), midecamycin, roxithromycin, kitasamycin or turimycin, josamycin, carbomycin or magnamycin, and/or spiramycin, and optionally the macrocyclic lactone antibiotic comprises ivermectin (or STROMECTOL™, SOOLANTRA™), or camostat mesilate (or FOIPAN™), and optionally the anti-viral drug or drug combination comprises daclatasvir (optionally DAKLINZA™);
and optionally the additional anti-viral drug or medication, or anti-microbial drug, is formulated with the chloroquine (for example, ARALEN™), chloroquine phosphate, quinine, chloroquine diphosphate, hydroxychloroquine (for example, PLAQUENIL™), lopinavir, ritonavir (or NORVIR™) and/or oseltamivir or is formulated separately from the chloroquine (for example, ARALEN™), chloroquine phosphate, quinine, chloroquine diphosphate, hydroxychloroquine (for example, PLAQUENIL™), lopinavir, ritonavir and/or oseltamivir,
and optionally the additional anti-viral drug or medication, or anti-microbial drug, is formulated with an immunosuppressive drug (optionally tocilizumab or atlizumab, or ACTEMRA™, or ROACTEMRA™),
and optionally the anti-viral drug or medication, or anti-microbial drug, or palliative agent comprises zinc (optionally a zinc sulphate, acetate, gluconate or picolinate, optionally administered at about 50 to 200 mg/day, or 100 mg/day) or at least one vitamin, wherein optionally the at least one vitamin comprises vitamin D, vitamin B12, vitamin B6 (or pyridoxine), vitamin K, vitamin E, and/or vitamin C (optionally administered at 500 mg bid), or a flavonoid, plant flavonol or quercetin optionally administered at between about 250 to 500 mg bid, or melatonin, which optionally is given or formulated for administration enterally or parenterally.

5. The product of manufacture of claim 1 or claim 2, wherein the drug or medication comprises: a therapeutic combination of drugs or a drug, a pharmaceutical dosage form, a drug delivery device, or a product of manufacture, comprising:
(a) oseltamivir, lopinavir and ritonavir; optionally further comprising chloroquine or hydroxychloroquine; further optionally comprising opaganib; and further optionally comprising zinc and bismuth; optionally wherein the lopinavir, ritonavir and oseltamivir are formulated or packaged for administration as or dosing in the ratio of 25:100:75 of lopinavir:ritonavir:oseltamivir;
(b) lopinavir, ritonavir, and chloroquine or hydroxychloroquine; optionally further comprising remdesivir and interferon;
(c) colchicine; optionally further comprising hydroxychloroquine; optionally further comprising zinc, and optionally further comprising an avermectin class antibiotic and vitamin C and/or vitamin D; or
(d) hydroxychloroquine and an antibiotic, optionally further comprising zinc and vitamin C and/or vitamin D; optionally wherein the antibiotic is selected from the group consisting of azithromycin, doxycycline, and clarithromycin.

6. The product of manufacture of any of the preceding claims, wherein the drug or medication comprises:
(a) a chloroquine (for example, ARALEN™), chloroquine phosphate, quinine, chloroquine diphosphate, hydroxychloroquine (for example, PLAQUENIL™), lopinavir, ritonavir (optionally NORVIR™) and/or oseltamivir formulated separately or together, or the lopinavir and ritonavir are formulated together and the oseltamivir is formulated separately; or
(b) a therapeutic combination of drugs or drug, pharmaceutical dosage form, wherein the chloroquine (for example, ARALEN™), chloroquine phosphate, quinine, chloroquine diphosphate, hydroxychloroquine (for example, PLAQUENIL™), lopinavir, ritonavir (optionally NORVIR™) and/or oseltamivir and/or the anti-viral drug or medication, or anti-microbial drug are formulated or contained in a liquid formulation (optionally sterile saline or water), a spray, a powder, an aerosol, or any formulation for inhalation.

7. A method for treating, preventing, ameliorating, slowing the progress of, decreasing the severity of or preventing a microbial infection in an individual in need thereof comprising administering a medication or a drug combination as contained in a product of manufacture fabricated or manufactured as an inhaler, asthma “puffer-like” device, nebulizer or nasal spray device or equivalent, for delivering a medication or a drug, as set forth in any of claims 1 to 6, or any of the preceding claims,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

8. A product of manufacture fabricated or manufactured as an inhaler, asthma “puffer-like” device, nebulizer or nasal spray device or equivalent, for delivering a medication or a drug, as set forth in any of claims 1 to 6, for use in treating, preventing, ameliorating, slowing the progress of, decreasing the severity of or preventing a microbial infection in an individual in need thereof,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

9. Use of a product of manufacture fabricated or manufactured as an inhaler, asthma “puffer-like” device, nebulizer or nasal spray device or equivalent, for delivering a medication or a drug, as set forth in any of claims 1 to 6, in the manufacture of a medicament for treating, preventing, ameliorating, slowing the progress of, decreasing the severity of or preventing a microbial infection in an individual in need thereof,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

10. The method of claim 7, product of manufacture of claim 8, or use of claim 9, wherein the individual in need thereof suffers from long term effects, or chronic effects or symptoms, of a viral infection, or the individual in need thereof has not fully recovered from the viral infection weeks or even months after first experiencing symptoms, or the individual in need thereof experiences continuous symptoms for weeks or months after being first diagnosed or treated with the viral infection, or the individual in need thereof feels better for weeks, then relapses with old or new symptoms,
and optionally the medication or the drug combination is administered to prevent a so-called “long-hauler” syndrome, or to treat or prevent continuous symptoms for weeks or months, or to prevent or treat relapsing with old or new symptoms,
wherein optionally the viral infection is a COVID-19 infection.

11. Use of a product of manufacture fabricated or manufactured as an inhaler, asthma “puffer-like” device, nebulizer or nasal spray device or equivalent, as set forth in any of claims 1 to 6, for delivering a medication or a drug,
wherein optionally the delivering of the medication or the drug is for treating, preventing, ameliorating, slowing the progress of, decreasing the severity of or preventing a microbial infection in an individual in need thereof comprising administering a medication or a drug combination,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.

12. A product of manufacture fabricated or manufactured as an inhaler, asthma “puffer-like” device, nebulizer or nasal spray device or equivalent, as set forth in any of claims 1 to 6, for use in delivering a medication or a drug,
wherein optionally the delivering of the medication or the drug is for treating, preventing, ameliorating, slowing the progress of, decreasing the severity of or preventing a microbial infection in an individual in need thereof comprising administering a medication or a drug combination,
wherein the microbial infection is a bacterial or a viral infection, and optionally the viral infection is caused by a respiratory virus, and optionally the respiratory virus is an influenza virus, respiratory syncytial virus, parainfluenza virus, adenovirus, rhinovirus, human metapneumovirus, hantaviruses, enterovirus or a coronavirus infection, and optionally the coronavirus is a COVID-19 virus,
and optionally the infection is caused by a virus in the subfamily Orthocoronavirinae, or a virus in the family Coronaviridae, or a virus in the order Nidovirales.
